(19) United States
(12) Patent Application Publication
(10) Pub. No.: US 2023/0243854 A1
(43) Pub. Date: Aug. 3, 2023

(54) IMMUNOASSAYS FOR DETECTING WILD TYPE HUNTINGTIN PROTEIN AND METHODS OF TREATMENT EMPLOYING SUCH IMMUNOASSAYS

(71) Applicant: WAVE LIFE SCIENCES LTD., Singapore (SG)

(72) Inventors: Ramakrishna Bharadwaj Boyanapalli, Cambridge, MA (US); Jaya Goyal, Cambridge, MA (US)

(73) Assignee: WAVE LIFE SCIENCES LTD., Singapore (SG)

(21) Appl. No.: 18/174,127

(22) Filed: Feb. 24, 2023

Related U.S. Application Data
(63) Continuation of application No. PCT/US2021/047277, filed on Aug. 24, 2021.
(60) Provisional application No. 63/167,395, filed on Mar. 29, 2021.
(60) Provisional application No. 63/069,647, filed on Aug. 24, 2020.

(51) Int. Cl. G01N 33/68

(52) U.S. Cl. CPC G01N 33/6896; G01N 2800/2835; G01N 2800/52

(57) ABSTRACT

The present disclosure relates to immunoassay methods and immunoassay kits for determining measuring the amount of wild type huntingtin protein in a sample, e.g., a cerebral spinal fluid sample from a Huntington's disease patient, as well as methods of treating Huntington's disease patients employing such immunoassays.

### CROSS REFERENCE TO RELATED APPLICATIONS

This application is a continuation of International Application No. PCT/US2021/047277, filed Aug. 24, 2021, which claims the benefit of U.S. Provisional Application No. 63/069,647, filed Aug. 24, 2020, and U.S. Provisional Application No. 63/167,395, filed Mar. 29, 2021, the disclosures of each of which are incorporated herein by reference in its entirety.

### 1. TECHNICAL FIELD

The present disclosure relates to immunoassay methods and immunoassay kits for determining the amount of wild type huntingtin protein in a sample, e.g., a cerebral spinal fluid sample, from a Huntington's disease patient, as well as methods of treating Huntington's disease patients employing such immunoassays.

### 2. BACKGROUND

Wild type huntingtin protein (wtHTT) plays an important role in embryogenesis and survival of neurons in the adult forebrain. An autosomal dominant mutation in at least one of the two alleles of huntingtin (HTT) gene can lead to Huntington's disease (HD), an inherited neurodegenerative disease. This pathogenic gain-of-function mutation of HTT gene, often seen in exon 1 of the HTT gene, is an expansion of the trinucleotide (CAG) repeat to 36 times or more in the HTT gene. The expanded CAG repeat has shown to be detrimental to neurons. The length of the expanded HTT CAG repeat segment determines the age of HD on-set. Moreover, the severity of HD can be indicated by the concentration of mutant huntingtin protein (mHTT) in the cerebral spinal fluid of the patient.

There are both non-allele-specific and allele-specific therapeutic candidates that either indiscriminately lower the amounts of both wtHTT and mHTT in HD patients, or selectively lower the amount of mHTT in HD patients, respectively. The extent of wtHTT-lowering by these agents, however, has not been evaluated because no assay is currently available for determining the level of wtHTT in patients.

### 3. SUMMARY OF THE INVENTION

The present disclosure relates to methods and kits for determining the amount of wtHTT in a sample, e.g., a cerebral spinal fluid (CSF) sample.

In one aspect, the present disclosure provides a method of determining the amount of wtHTT in a sample, the method comprising: (a) determining a first amount of total huntingtin protein (tHTT) in the sample; (b) contacting the sample with an mHTT-specific antibody composition comprising an mHTT antibody, wherein the concentration of the mHTT-specific antibody is a function of the first amount of the tHTT; (c) removing the mHTT-specific antibody composition from the sample; and (d) determining the amount of wtHTT protein in the sample by determining a second amount of tHTT in the sample.

In certain embodiments, when the first amount of tHTT is at least about 15 fM, the concentration of the mHTT-specific antibody is between about 0.01 ng/ml and about 1000 ng/ml, between about 1 ng/ml and about 100 ng/ml, between about 10 ng/ml and about 100 ng/ml, between about 100 ng/ml and about 1000 ng/ml, between about 1 ng/ml and about 10 ng/ml, between about 1 ng/ml and about 5 ng/ml, or between about 1.25 ng/ml and about 5 ng/ml of the sample.

In certain embodiments, the first and the second amounts of tHTT are determined by an immunoassay comprising an anti-huntingtin protein (anti-HTT) antibody. In certain embodiments, the anti-HTT antibody can bind to both wtHTT and mHTT. In certain embodiments, the anti-HTT antibody is attached to a label. In certain embodiments, the immunoassay comprises a secondary antibody that binds to the anti-HTT antibody, wherein the secondary antibody is attached to a label. In certain embodiments, the label is a chemiluminescent molecule, a fluorochromes, a colored molecules, a radioisotope, or a combination thereof.

In certain embodiments, the mHTT-specific antibody composition further comprises particles. In certain embodiments, the particles are agarose particles or magnetic particles. In certain embodiments, the mHTT specific antibody is an anti-polyglutamine antibody. In certain embodiments, the mHTT specific antibody is MW1. In certain embodiments, the particles are linked to the mHTT specific antibody. In certain embodiments, the particles are linked to the mHTT specific antibody through antibody binding proteins, streptavidin-biotin interaction, or covalent immobilization. In certain embodiments, the particles are linked to the mHTT specific antibody through streptavidin-biotin interaction, wherein the particles are attached to streptavidin, and the mHTT specific antibody is in biotinylated form.

In certain embodiments, the step (c) further comprises removing the mHTT-specific antibody composition from the sample by centrifugation, wherein the particles are agarose particles. In certain embodiments, the step (c) further comprises removing the mHTT-specific antibody composition from the sample by applying a magnetic field to the sample, wherein the particles are magnetic particles.

In certain embodiments, the sample is a cerebral spinal fluid sample. In certain embodiments, the sample is from a subject having Huntington's disease. In certain embodiments, the subject has received or is receiving a treatment for Huntington's disease.

In one aspect, the present disclosure provides a kit for of determining the amount of wild type huntingtin protein (wtHTT) in a sample, the kit comprising: a mutant huntingtin protein (mHTT)-specific antibody composition comprising an mHTT antibody; an anti-huntingtin protein (anti-HTT) antibody; and instructions for of determining the amount of wild type huntingtin protein (wtHTT) in the sample, wherein the instructions comprise directions to (a) determining a first amount of total huntingtin protein (tHTT) in the sample; (b) contacting the sample with a mutant huntingtin protein (mHTT)-specific antibody composition, wherein the concentration of the mHTT-specific antibody is a function of the first amount of the tHTT; (c) removing the mHTT-specific antibody composition from the sample; and (d) determining the amount of wtHTT protein in the sample by determining a second amount of tHTT in the sample.

In certain embodiments, when the first amount of tHTT is at least about 15 fM, the concentration of the mHTT-specific antibody is between about 0.01 ng/ml and about 1000 ng/ml, between about 1 ng/ml and about 100 ng/ml, between about 10 ng/ml and about 100 ng/ml, between about 100 ng/ml and about 1000 ng/ml, between about 1 ng/ml and about 10 ng/ml, between about 1 ng/ml and about 5 ng/ml, or between about 1.25 ng/ml and about 5 ng/ml of the sample.

In certain embodiments, the first and the second amounts of tHTT are determined by an immunoassay comprising the anti-HTT antibody. In certain embodiments, the anti-HTT antibody can bind to both wtHTT and mHTT.

In certain embodiments, the anti-HTT antibody is attached to a label. In certain embodiments, the kits disclosed herein further comprises a secondary antibody that binds to the anti-HTT antibody, wherein the secondary antibody is attached to a label. In certain embodiments, the label is a chemiluminescent molecule, a fluorochromes, a colored molecules, a radioisotope, or a combination thereof.

In certain embodiments, the mHTT-specific antibody composition further comprises particles. In certain embodiments, the particles are agarose particles or magnetic particles. In certain embodiments, the mHTT specific antibody is an anti-polyglutamine antibody.

In certain embodiments, the particles are linked to the mHTT specific antibody. In certain embodiments, the particles linked to the mHTT specific antibody through antibody binding proteins, streptavidin-biotin interaction, or covalent immobilization. In certain embodiments, the particles are linked to the mHTT specific antibody through streptavidin-biotin interaction, wherein the particles are attached to streptavidin, and the mHTT specific antibody is in biotinylated form.

In certain embodiments, the step (c) further comprises removing the mHTT-specific antibody composition from the sample by centrifugation, wherein the particles are agarose particles. In certain embodiments, the step (c) further comprises removing the mHTT-specific antibody composition from the sample by applying a magnetic field to the sample, wherein the particles are magnetic particles.

In certain embodiments, the sample is a cerebral spinal fluid sample. In certain embodiments, the sample is from a subject having Huntington's disease. In certain embodiments, the subject has received or is receiving a treatment for Huntington's disease.

In one aspect, the present disclosure provides a method of treating a subject, the method comprising: a) determining the amount of wild type huntingtin protein (wtHTT) in a cerebral spinal fluid sample from the subject, wherein determining comprises: (i) determining a first amount of total huntingtin protein (tHTT) in the sample; (ii) contacting the sample with a mutant huntingtin protein (mHTT)-specific antibody composition comprising an mHTT-specific antibody, wherein the concentration of the mHTT-specific antibody is a function of the first amount of the tHTT; (iii) removing the mHTT-specific antibody composition from the sample; and (iv) determining the amount of wtHTT in the sample by determining a second amount of tHTT in the sample; and b) administering to the subject a treatment for Huntington's disease if the amount of wtHTT determined in step (a) is decreased as compared to a reference value.

In one aspect, the present disclosure provides method of adjusting a treatment for Huntington's disease of a subject that has received or is receiving the treatment, the method comprising: a) determining the amount of wild type huntingtin protein (wtHTT) in a cerebral spinal fluid sample from the subject, wherein determining comprises: (i) determining a first amount of total huntingtin protein (tHTT) in the sample; (ii) contacting the sample with a mutant huntingtin protein (mHTT)-specific antibody composition comprising an mHTT-specific antibody, wherein the concentration of the mHTT-specific antibody is a function of the first amount of the tHTT; (iii) removing the mHTT-specific antibody composition from the sample; and (iv) determining the amount of wtHTT in the sample by determining a second amount of tHTT in the sample; and b) continuing administering the treatment to the subject if the amount of wtHTT determined in step (a) is unchanged or decreased as compared to a reference value.

In certain embodiments, when the first amount of tHTT is at least about 15 fM, the concentration of the mHTT-specific antibody is between about 0.01 ng/ml and about 1000 ng/ml, between about 1 ng/ml and about 100 ng/ml, between about 10 ng/ml and about 100 ng/ml, between about 100 ng/ml and about 1000 ng/ml, between about 1 ng/ml and about 10 ng/ml, between about 1 ng/ml and about 5 ng/ml, or between about 1.25 ng/ml and about 5 ng/ml of the sample.

In certain embodiments, the first and the second amounts of tHTT are determined by an immunoassay comprising an anti-huntingtin protein (anti-HTT) antibody. In certain embodiments, the anti-HTT antibody can bind to both wtHTT and mHTT. In certain embodiments, the anti-HTT antibody is attached to a label. In certain embodiments, the immunoassay comprises a secondary antibody that binds to the anti-HTT antibody, wherein the secondary antibody is attached to a label. In certain embodiments, the label is a chemiluminescent molecule, a fluorochromes, a colored molecules, a radioisotope, or a combination thereof.

In certain embodiments, the mHTT-specific antibody composition further comprises particles. In certain embodiments, the particles are agarose particles or magnetic particles. In certain embodiments, the mHTT specific antibody is an anti-polyglutamine antibody. In certain embodiments, the mHTT specific antibody is MW1. In certain embodiments, the particles are linked to the mHTT specific antibody. In certain embodiments, the particles are linked to the mHTT specific antibody through antibody binding proteins, streptavidin-biotin interaction, or covalent immobilization. In certain embodiments, the particles are linked to the mHTT specific antibody through streptavidin-biotin interaction, wherein the particles are attached to streptavidin, and the mHTT specific antibody is in biotinylated form.

In certain embodiments, the step (a)-(iii) further comprises removing the mHTT-specific antibody composition from the sample by centrifugation, wherein the particles are agarose particles. In certain embodiments, the step (a)-(iii) further comprises removing the mHTT-specific antibody composition from the sample by applying a magnetic field to the sample, wherein the particles are magnetic particles. In certain embodiments, the reference value is an amount of wtHTT of subjects not having Huntington's disease.

### 4. BRIEF DESCRIPTION OF THE DRAWINGS

FIG. 1 is a schematic showing of an exemplary embodiment of the methods disclosed herein for determining the amount of wtHTT in a sample.

FIG. 2 shows the efficiency of different concentrations of MW1 beads in depleting mHTT in artificial CSF spiked with 50 fM wtHTT and mHTT.

FIG. 3 shows the efficiency of MW1 beads in depleting mHTT in non-HD human CSF with 70 fM endogenous wtHTT and spiked with various amounts of mHTT.

FIG. 4 shows the effects of free MW1 (not bound to beads) on the tHTT and mHTT assays.

FIG. 5 shows assay optimization results in artificial CSF. tHTT assay results were shown.

FIG. 6 shows assay optimization results in artificial CSF. mHTT assay results were shown.

FIG. 7 shows assay optimization results in pooled non-HD human CSF.

FIG. 8 shows the measurements of wtHTT and tHtt levels in the CSFs of HD disease patients.

FIG. 9 shows an example of 96 well plate map.

FIG. 10 is a schematic showing of the study design of the PRECISION-HD clinical studies.

FIG. 11 is a table showing the patient dispositions in PRECISION-HD2 clinical study.

FIG. 12 is a table showing the patient demographics and HD disease history in PRECISION-HD2 clinical study.

FIG. 13 shows wtHTT levels measured in the HD patients at the PRECISION-HD2 core study.

FIG. 14 shows wtHTT levels measured in the HD patients at the PRECISION-HD2 OLE (open label extension) study.

FIG. 15 shows wtHTT levels measured in the HD patients at the PRECISION-HD1 core study.

FIG. 16 shows wtHTT levels measured in the HD patients at the PRECISION-HD1 OLE (open label extension) study.

### 5. DETAILED DESCRIPTION

The present disclosure provides methods and kits for determining the amount of wtHTT in a sample. Currently, no assays are available to quantify wtHTT in a sample. While assays are available to determine the amounts of mHTT or total huntingtin protein (tHTT), the complexity of these assays and the nonequivalence of values generated by these assays prevent the use of these assays to deduce the amount of wtHTT in a sample. The present disclosure provide a novel assay scheme to determine the amount of wtHTT in a sample (e.g., a CSF sample from a HD patient). The present disclosure is partly based on the use of an mHTT specific antibody composition at selected concentrations to selectively and effectively deplete mHTT from a sample (e.g., a CSF sample) that contains both wtHTT and mHTT, while leaving wtHTT unaltered in the sample. The concentrations of the mHTT specific antibody composition are selected based on the range and model of tHTT concentrations seen in samples obtained from subjects (e.g. HD patients).

Non-limiting embodiments of the present disclosure are described by the present specification and Examples.

For purposes of clarity of disclosure and not by way of limitation, the detailed description is divided into the following subsections:
5.1 Definitions;
5.2 List of abbreviations;
5.3 Methods for determining the amount of wtHTT;
5.4 Kits;
5.5 Methods of treatment; and
5.6 Exemplary non-limiting embodiments

### 5.1 Definitions

The terms used in this specification generally have their ordinary meanings in the art, within the context of this disclosure and in the specific context where each term is used. Certain terms are discussed below, or elsewhere in the specification, to provide additional guidance to the practitioner in describing the compositions and methods of the disclosure and how to make and use them.

As used herein, the use of the word “a” or “an” when used in conjunction with the term “comprising” in the claims and/or the specification may mean “one,” but it is also consistent with the meaning of “one or more,” “at least one,” and “one or more than one.” Still further, the terms “having,” “including,” “containing” and “comprising” are interchangeable and one of skill in the art is cognizant that these terms are open ended terms.

The term “about” or “approximately” means within an acceptable error range for the particular value as determined by one of ordinary skill in the art, which will depend in part on how the value is measured or determined, i.e., the limitations of the measurement system. For example, “about” can mean within 3 or more than 3 standard deviations, per the practice in the art. Alternatively, “about” can mean a range of up to 20%, preferably up to 10%, more preferably up to 5%, and more preferably still up to 1% of a given value. Alternatively, particularly with respect to biological systems or processes, the term can mean within an order of magnitude, preferably within 5-fold, and more preferably within 2-fold, of a value.

An “individual” or “subject” herein is a vertebrate, such as a human or non-human animal, for example, a mammal. Mammals include, but are not limited to, humans, non-human primates, farm animals, sport animals, rodents and pets. Non-limiting examples of non-human animal subjects include rodents such as mice, rats, hamsters, and guinea pigs; rabbits; dogs; cats; sheep; pigs; goats; cattle; horses; and non-human primates such as apes and monkeys.

As used herein, the term “disease” refers to any condition or disorder that damages or interferes with the normal function of a cell, tissue, or organ.

As used herein, and as well-understood in the art, “treatment” is an approach for obtaining beneficial or desired results, including clinical results. For purposes of this subject matter, beneficial or desired clinical results include, but are not limited to, alleviation or amelioration of one or more sign or symptoms, diminishment of extent of disease, stabilized (i.e., not worsening) state of disease, prevention of disease, delay or slowing of disease progression, and/or amelioration or palliation of the disease state. The decrease can be a 10%, 20%, 30%, 40%, 50%, 60%, 70%, 80%, 90%, 95%, 98% or 99% decrease in severity of complications or symptoms. “Treatment” can also mean prolonging survival as compared to expected survival if not receiving treatment.

5.2 List of abbreviations
aCSF: artificial cerebrospinal fluid
artificial CSF: artificial cerebrospinal fluid
ASO: antisense oligonucleotide
BSA: bovine serum albumin
CAG: cytosine-adenine-guanine
CNS: central nervous system
CSF: cerebrospinal fluid
ELISA: enzyme-linked immunosorbent assay

### fM: Femtomolar

HD: Huntington's disease
HTT, HTT: huntingtin, Huntingtin

### INN: International Nonproprietary Name

### IT: Intrathecal

### IV: Intravenous

KD: knockdown
mHTT: mutant huntingtin protein
ng/ml: nanogram per milliliter
PBS: phosphate buffered saline
phCSF: pooled non-HD human CSF
tHTT: total huntingtin protein
μg/μl: microgram per microliter
wtHTT: wild type huntingtin protein
5.3 Methods for Determining the Amount of wtHTT

The present disclosure provides methods for determining the amount of wtHTT in a sample. The methods disclosed herein comprise: (a) determining a first amount of tHTT in the sample; (b) contacting the sample with a mHTT-specific antibody composition comprising an mHTT-specific antibody, wherein the concentration of the mHTT-specific antibody composition is a function of the first amount of the tHTT; (c) removing the mHTT-specific antibody composition from the sample; and (d) determining the amount of wtHTT in the sample by determining a second amount of tHTT in the sample.

In certain embodiments, the methods disclosed herein comprise (a) contacting the sample with a mHTT-specific antibody composition comprising an mHTT-specific antibody, wherein the concentration of the mHTT-specific antibody composition is a function of a first amount of the tHTT determined in the sample; (b) removing the mHTT-specific antibody composition from the sample; and (c) determining the amount of wtHTT in the sample by determining a second amount of tHTT in the sample.

In the presently disclosed methods, the concentration of the mHTT-specific antibody needs to be high enough to be able to effectively deplete all or a majority of mHTT from the sample, yet cannot be too high to unselectively bind to and pull down wtHTT from the sample. Moreover, the initial amount of tHTT (e.g., a combination of endogenous wtHTT and mHTT) in a sample needs to be in certain range for the mHTT-specific antibody composition to selectively and effectively deplete the mHTT in the sample. In certain embodiments, when the first amount of tHTT is at least about 15 fM, the concentration of the mHTT-specific antibody is between about 0.01 ng/ml and about 1000 ng/ml, between about 0.01 ng/ml and about 100 ng/ml, between about 0.1 ng/ml and about 10 ng/ml, between about 0.01 ng/ml and about 1 ng/ml, between about 0.01 ng/ml and about 0.1 ng/ml, between about 0.1 ng/ml and about 1000 ng/ml, between about 0.1 ng/ml and about 100 ng/ml, between about 0.1 ng/ml and about 10 ng/ml, between about 0.1 ng/ml and about 1 ng/ml, between about 1 ng/ml and about 1000 ng/ml, between about 1 ng/ml and about 100 ng/ml, between about 10 ng/ml and about 1000 ng/ml, between about 10 ng/ml and about 100 ng/ml, between about 100 ng/ml and about 1000 ng/ml, between about 1 ng/ml and about 10 ng/ml, between about 5 ng/ml and about 10 ng/ml, between about 1 ng/ml and about 5 ng/ml, or between about 1.25 ng/ml and about 5 ng/ml of the sample. In certain embodiments, when the first amount of tHTT is at least about 15 fM, at least about 20 fM, at least about 40 fM, at least about 60 fM, at least about 80 fM, at least about 100 fM, at least about 150 fM, at least about 200 fM, at least about 250 fM, at least about 300 fM, at least about 350 fM, at least about 400 fM or more, the concentration of the mHTT-specific antibody is between about 0.01 ng/ml and about 1000 ng/ml, between about 0.01 ng/ml and about 100 ng/ml, between about 0.1 ng/ml and about 10 ng/ml, between about 0.01 ng/ml and about 1 ng/ml, between about 0.01 ng/ml and about 0.1 ng/ml, between about 0.1 ng/ml and about 1000 ng/ml, between about 0.1 ng/ml and about 100 ng/ml, between about 0.1 ng/ml and about 10 ng/ml, between about 0.1 ng/ml and about 1 ng/ml, between about 1 ng/ml and about 1000 ng/ml, between about 1 ng/ml and about 100 ng/ml, between about 10 ng/ml and about 1000 ng/ml, between about 10 ng/ml and about 100 ng/ml, between about 100 ng/ml and about 1000 ng/ml, between about 1 ng/ml and about 10 ng/ml, between about 5 ng/ml and about 10 ng/ml, between about 1 ng/ml and about 5 ng/ml, or between about 1.25 ng/ml and about 5 ng/ml of the sample. In certain embodiments, when the first amount of tHTT is at least about 15 fM, the concentration of the mHTT-specific antibody is about 0.01 ng/ml, about 0.1 ng/ml, about 1 ng/ml, about 10 ng/ml, about 50 ng/ml, about 100 ng/ml, about 500 ng/ml, about 1000 ng/ml, about 1.25 ng/ml, about 1.5 ng/ml, about 1.75 ng/ml, about 2 ng/ml, about 2.5 ng/ml, about 3 ng/ml, about 3.5 ng/ml, about 4 ng/ml, about 4.5 ng/ml, or about 5 ng/ml of the sample.

In certain embodiments, the step of removing the mHTT-specific antibody composition from the sample removes all or a majority of mHTT from the sample. In certain embodiments, removing the mHTT-specific antibody composition from the sample removes at least about 70%, about 75%, about 80%, about 85%, about 90%, about 95%, about 99% or more of mHTT from the sample. In certain embodiments, removing the mHTT-specific antibody composition from the sample removes about 100% of mHTT from the sample.

In certain embodiments, the first and the second amounts of tHTT are determined by an immunoassay. Any suitable immunoassays known in the art for determining the amount of tHTT can be used with the presently disclosed methods. Non-limiting suitable examples of immunoassays that can be used with the presently disclosed methods include ELISA, sandwich immunoassay, radioimmunoassay, immunoblot or Western blot, and an array. In certain embodiments, the immunoassay is a single molecule counting assay (SMC) (e.g., an immunoassay performed on SMC Singulex Erenna instrument). In certain embodiments, the immunoassay is a meso scale discovery (MSD) based assay. In certain embodiments, the immunoassay comprises an anti-HTT antibody. In certain embodiments, the anti-HTT antibody indiscriminately binds to wtHTT and mHTT. Any suitable anti-HTT antibodies known in the art that indiscriminately binds to wtHTT and mHTT can be used with the presently disclosed methods. Non-limiting examples of suitable anti-HTT antibodies that can be used with the presently disclosed methods include 2B7 and DF7. In certain embodiments, the immunoassay is performed by contacting a sample comprising HTT (e.g., including wtHTT and mHTT) with the anti-HTT antibody under conditions effective to allow for formation of a complex between the anti-HTT antibody and the HTT. The method may occur in a solution, or the anti-HTT antibody or HTT may be immobilized on a solid surface. Non-limiting examples of suitable solid surfaces include microtiter plates, test tubes, beads, resins, and other polymers. The antibody-HTT complex is then detected and measured. Any suitable methods known in the art for detecting and measuring an amount of an antibody—polypeptide complex can be used with the presently disclosed methods. In certain embodiments, a suitable method for detecting and measuring an amount of an antibody—polypeptide complex is based on the detection of a label. In certain embodiments, the anti-HTT antibody is attached to a label. In certain embodiments, the immunoassay comprises a secondary antibody that specifically binds to the anti-HTT antibody, where the secondary antibody is attached to a label. Non-limiting examples of suitable labels that can be used with the presently disclosed methods include luminescent molecules, chemiluminescent molecules, fluorochromes, fluorescent quenching agents, colored molecules, radioisotopes, scintillant, biotin, avidin, streptavidin, protein A, protein G, polyhistidine, Ni2+, Flag tags, myc tags, heavy metals, and enzymes (e.g., alkaline phosphatase, peroxidase, and luciferase).

In certain embodiments, the methods disclosed herein further comprising determining the amount of mHTT in the sample. Any methods known in the art for determining mHTT can be used with the presently disclosed methods. In certain embodiments, the amount of mHTT is determined by an immunoassay. Any suitable immunoassays known in the art for determining the amount of mHTT can be used with the presently disclosed methods. In certain embodiments, anti-HTT antibodies and anti-mHTT antibodies are used for determining the amount of mHTT.

In certain embodiments, the mHTT-specific antibody composition comprises an mHTT-specific antibody and particles. Any suitable particles known in the art for immunoprecipitation can be used with the presently disclosed methods. In certain embodiments, the particles are magnetic particles (e.g., magnetic beads) or agarose particles (e.g., agarose resin, agarose beads). Any suitable magnetic particles or agarose particles known in the art can be used with the presently disclosed methods. Non-limiting examples of magnetic particles include Dynabeads, Pierce magnetic beads, Sigma-Millipore magnetic beads, and Promega magnetic beads. In certain embodiments, the mHTT-specific antibody is linked to the particles. Any suitable strategies known in the art for antibody immobilization can be used with the presently disclosed methods. In certain embodiments, the mHTT specific antibody is linked to the particles through antibody-binding proteins. Non-limiting examples of suitable antibody-binding proteins that can be used with the presently disclosed methods include Protein A, Protein G, Protein A/G and Protein L. In certain embodiments, the mHTT specific antibody is linked to the particles through streptavidin-biotin interaction, where the mHTT-specific antibody is in biotinylated format, and the particles are attached to streptavidin. In certain embodiments, the mHTT specific antibody is linked to the particles through covalent immobilization. Covalent immobilization strategies chemically bind the antibody to the particles and remove the requirement for Protein A/G-dependent antibody immobilization. Any suitable products known in the art that provide particles that react with primary amines (—NH2) on the antibody to permanently bind the antibody to the support can be used with the presently disclosed methods.

Any suitable mHTT-specific antibodies known in the art can be used with the presently disclosed methods. In certain embodiments, the mHTT-specific antibody is an anti-polyglutamine antibody. Non-limiting examples of mHTT-specific antibody include MW1, 3B5H10, mEM48 (e.g., Chemicon Cat #MAB5374), and MW7.

In certain embodiments, the mHTT-specific antibody composition is removed from the sample by centrifugation, wherein the particles are agarose particles. In certain embodiments, the mHTT-specific antibody composition is removed from the sample by applying a magnetic field to the sample, wherein the particles are magnetic particles.

In certain embodiments, the sample is a biological sample from a subject. In certain embodiments, the sample is a cerebral spinal fluid sample, a blood sample, a peripheral blood mononuclear cell sample, a urine sample, or cells that can express huntingtin protein. In certain embodiments, the sample is a cerebral spinal fluid sample. In certain embodiments, the sample is from a subject. In certain embodiment, the subject can be a human or a non-human subject, such as, but not limited to, a dog, a cat, a horse, a rodent, or a non-human primate.

In certain embodiments, the subject is a human subject. In certain embodiments, the subject has HD. In certain embodiments, the subject has received or is receiving a treatment for Huntington's disease. In certain embodiments, the subject has not received a treatment for Huntington's disease. In certain embodiments, the treatment lower the amounts of both wtHTT and mHTT in a subject. In certain embodiments, the treatment specifically lower mHTT without altering wtHTT in a subject.

In certain embodiments, the methods disclosed herein can be performed on a number of samples that have been collected from the subject at different times (e.g., different days, different times on the same day).

### 5.4 Kits.

The present disclosure provides kits for determining the amount of wild type huntingtin protein (wtHTT) in a sample, the kit comprising: a mutant huntingtin protein (mHTT)-specific antibody composition comprising an mHTT-specific antibody; and an anti-huntingtin protein (anti-HTT) antibody.

Any suitable anti-HTT antibodies known in the art that indiscriminately binds to wtHTT and mHTT can be used with the presently disclosed methods (e.g., anti-HTT antibodies disclosed in Section 5.3 of the present disclosure). In certain embodiments, the anti-HTT antibody is attached to a label. In certain embodiments, the kits disclosed herein further comprise a secondary antibody that specifically binds to the anti-HTT antibody, where the secondary antibody is attached to a label. Any suitable labels known in the art for immunoassay can be used with the presently disclosed kits (e.g., labels disclosed in Section 5.3 of the present disclosure).

In certain embodiments, the mHTT-specific antibody composition comprises an mHTT-specific antibody and particles. Any suitable particles known in the art for immunoprecipitation can be used with the presently disclosed kits (e.g., particles disclosed in Section 5.3 of the present disclosure). In certain embodiments, the mHTT-specific antibody is linked to the particles. Any suitable strategies known in the art for antibody immobilization can be used with the presently disclosed methods (e.g., strategies disclosed in Section 5.3 of the present disclosure)

Any suitable mHTT-specific antibodies known in the art can be used with the presently disclosed methods. In certain embodiments, the mHTT-specific antibody is an anti-polyglutamine (polyQ) antibody. Non-limiting examples of mHTT-specific antibody include MW1, 3B5H10, mEM48 (e.g., Chemicon Cat #MAB5374), and MW7.

In certain embodiments, the presently disclosed kits further comprise instructions for determining the amount of wtHTT in the sample. In certain embodiments, the instructions comprise methods as described in Section 5.3 of the present disclosure.

### 5.5 Methods of Treatment

The present disclosure provides methods of treating a subject, wherein the subject is diagnosed with Huntington's disease. In certain embodiments, the subject is determined to have a decreased amount of wtHTT as compared to a reference value in a sample of the subject. In certain embodiments, the amount of wtHTT is determined by the presently disclosed methods and kits (e.g., methods and kits disclosed in Sections 5.3 and 5.4 of the present disclosure respectively).

In certain embodiments, the presently disclosed methods for treating a subject comprise: a) determining the amount of wild type huntingtin protein (wtHTT) in a sample from the subject, wherein the determining comprises: (i) determining a first amount of total huntingtin protein (tHTT) in the sample; (ii) contacting the sample with a mutant huntingtin protein (mHTT)-specific antibody composition comprising an mHTT-specific antibody, wherein the concentration of the mHTT-specific antibody is a function of the first amount of the tHTT; (iii) removing the mHTT-specific antibody composition from the sample; and (iv) determining the amount of wtHTT in the sample by determining a second amount of tHTT in the sample; and b) administering to the subject a treatment for Huntington's disease if the amount of wtHTT determined in step (a) is decreased as compared to a reference value.

The present disclosure further provides methods of adjusting a treatment for Huntington's disease of a subject that has received or is receiving the treatment. In certain embodiments, the amount of wtHTT of the subject is monitored after or during the treatment. In certain embodiments, the treatment is adjusted based on the amount of wtHTT in a sample of the subject as compared to a reference value. In certain embodiments, the amount of wtHTT is determined by the presently disclosed methods and kits (e.g., methods and kits disclosed in Sections 5.3 and 5.4 of the present disclosure respectively).

In certain embodiments, the presently disclosed methods of adjusting a treatment for Huntington's disease of a subject that has received or is receiving the treatment comprise: a) determining the amount of wild type huntingtin protein (wtHTT) in a sample from the subject having Huntington's disease, wherein determining comprises: (i) determining a first amount of total huntingtin protein (tHTT) in the sample; (ii) contacting the sample with a mutant huntingtin protein (mHTT)-specific antibody composition comprising an mHTT-specific antibody, wherein the concentration of the mHTT-specific antibody is a function of the first amount of the tHTT; (iii) removing the mHTT-specific antibody composition from the sample; and (iv) determining the amount of wtHTT in the sample by determining a second amount of tHTT in the sample; and b) adjusting the treatment of the subject based on the amount of wtHTT determined in step (a). In certain embodiments, the methods comprise continuing administering the treatment if the amount of wtHTT determined in step (a) is unchanged or decreased as compared to a reference value. In certain embodiments, the methods comprise continuing administering the treatment if the amount of wtHTT determined in step (a) is unchanged, wherein the treatment is an allele-specific treatment. In certain embodiments, the methods comprise continuing administering the treatment if the amount of wtHTT determined in step (a) is decreased as compared to a reference value, wherein the treatment is a non-allele-specific treatment.

In certain embodiments, the reference value is the amount of wtHTT from heathy subjects. In certain embodiments, the reference value is the amount (e.g., mean or median) of wtHTT from subjects do not have Huntington's Disease. In certain embodiments, the reference value is a median or a mean value. In certain embodiments, the reference value is the amount of wtHTT from a sample of the same subject collected at an earlier timepoint, for example, an earlier timepoint before receiving the treatment for Huntington's disease or during the treatment for Huntington's disease.

Any suitable treatment methods for Huntington's disease known in the art can be used with the presently disclosed methods and kits. In certain embodiments the treatment comprises, but not limited to psychotherapy, speech therapy, physical therapy, occupational therapy, therapy with medication for movement disorders, therapy with medication for psychiatric disorders and combinations thereof. In certain embodiments the therapy with medication for movement disorders comprises, but not limited to drugs to control movement, antipsychotic drugs and combinations thereof. In certain embodiments, the therapy with medication for psychiatric disorders comprises, but not limited to antidepressants, antipsychotic drugs, mood-stabilizing drugs and combinations thereof.

In certain embodiments, the treatment for Huntington's disease comprises a therapeutic agent, which lowers the amount of mHTT. In certain embodiments, the treatment is an allele-specific treatment which selectively lowers the amount of mHTT. In certain embodiments, the treatment is a non-allele-specific treatment which lowers both the amount of mHTT and the amount of wtHTT. Any suitable mHTT lowering therapeutic agents can be used with the present disclosure, for example the mHTT lowering therapeutic agents disclosed in Tabrizi et al., Neuron (2019); 101:801-819, the contents of which are incorporated herein by reference. In certain embodiments the treatment for Huntington's disease comprises but not limited to DNA-targeting agents, RNA-targeting agents, small molecules, treatments targeting alternative toxic species in HD, protein clearance strategies and combinations thereof. In certain embodiments the DNA-targeting agents comprise, but not limited to, zinc-finger nucleases (ZFNs), transcription activator-like effector nucleases (TALENs), Cas9 or other RNA-guided bacterial nucleases and combinations thereof. In certain embodiments the RNA-targeting agents comprise, but not limited to, RNA interference (RNAi), antisense oligonucleotides (ASOs), and small-molecule modulators of RNA processing and combinations thereof. In certain embodiments, the protein clearance strategies comprise, but not limited to, small-molecule therapeutic agents, proteolysis-targeting chimera proteins (PROTACs) to selectively tag specific proteins for ubiquitin-proteasome system (UPS) degradation and combinations thereof.

In certain embodiments, the sample is a biological sample from a subject. In certain embodiments, the sample is a cerebral spinal fluid sample, a blood sample, a peripheral blood mononuclear cell sample, a urine sample, or cells that can express huntingtin protein. In certain embodiments, the sample is a cerebral spinal fluid sample.

### 5.6 Exemplary Non-Limiting Embodiments

A. A method of determining the amount of wild type huntingtin protein (wtHTT) in a sample, the method comprising:

(a) determining a first amount of total huntingtin protein (tHTT) in the sample;

(b) contacting the sample with a mutant huntingtin protein (mHTT)-specific antibody composition comprising an mHTT-specific antibody, wherein the concentration of the mHTT-specific antibody is a function of the first amount of the tHTT;

(c) removing the mHTT-specific antibody composition from the sample; and

(d) determining the amount of wtHTT in the sample by determining a second amount of tHTT in the sample.

A1. The method of A, wherein when the first amount of tHTT is at least about 15 fM, the concentration of the mHTT-specific antibody is between about 0.01 ng/ml and about 1000 ng/ml, between about 1 ng/ml and about 100 ng/ml, between about 10 ng/ml and about 100 ng/ml, between about 100 ng/ml and about 1000 ng/ml, between about 1 ng/ml and about 10 ng/ml, between about 1 ng/ml and about 5 ng/ml, or between about 1.25 ng/ml and about 5 ng/ml of the sample.

A2. The method of A or A1, wherein the first and the second amounts of tHTT are determined by an immunoassay comprising an anti-huntingtin protein (anti-HTT) antibody.

A3. The method of A2, wherein the anti-HTT antibody can bind to both wtHTT and mHTT.

A4. The method of A2 or A3, wherein the anti-HTT antibody is attached to a label.

A5. The method of A2 or A3, wherein the immunoassay comprises a secondary antibody that binds to the anti-HTT antibody, wherein the secondary antibody is attached to a label.

A6. The method of any one of A2-A5, wherein the label is a chemiluminescent molecule, a fluorochromes, a colored molecules, a radioisotope, or a combination thereof.

A7. The method of any one of A-A6, wherein the mHTT-specific antibody composition further comprises particles.

A8. The method of A7, wherein the particles are agarose particles or magnetic particles.

A9. The method of any one of A-A8, wherein the mHTT specific antibody is an anti-polyglutamine antibody.

A10. The method of any one of A7-A9, wherein the particles are linked to the mHTT specific antibody.

A11. The method of A10, wherein the particles are linked to the mHTT specific antibody through antibody binding proteins, streptavidin-biotin interaction, or covalent immobilization.

A12. The method of A10 or A11, wherein the particles are linked to the mHTT specific antibody through streptavidin-biotin interaction, wherein the particles are attached to streptavidin, and the mHTT specific antibody is in biotinylated form.

A13. The method of any one of A8-A12, wherein the step (c) further comprises removing the mHTT-specific antibody composition from the sample by centrifugation, wherein the particles are agarose particles.

A14. The method of any one of A8-A12, wherein the step (c) further comprises removing the mHTT-specific antibody composition from the sample by applying a magnetic field to the sample, wherein the particles are magnetic particles.

A15. The method of any one of A-A14, wherein the sample is a cerebral spinal fluid sample.

A16. The method of any one of A-A15, wherein the sample is from a subject having Huntington's disease.

A17. The method of A16, wherein the subject has received or is receiving a treatment for Huntington's disease.

B. A kit for of determining the amount of wild type huntingtin protein (wtHTT) in a sample, the kit comprising:

a mutant huntingtin protein (mHTT)-specific antibody composition comprising an mHTT-specific antibody;

an anti-huntingtin protein (anti-HTT) antibody; and

instructions for of determining the amount of wild type huntingtin protein (wtHTT) in the sample, wherein the instructions comprise directions to (a) determining a first amount of total huntingtin protein (tHTT) in the sample; (b) contacting the sample with a mutant huntingtin protein (mHTT)-specific antibody composition, wherein the concentration of the mHTT-specific antibody is a function of the first amount of the tHTT; (c) removing the mHTT-specific antibody composition from the sample; and (d) determining the amount of wtHTT protein in the sample by determining a second amount of tHTT in the sample.

B1. The kit of B, wherein when the first amount of tHTT is at least about 15 fM, the concentration of the mHTT-specific antibody is between about 0.01 ng/ml and about 1000 ng/ml, between about 1 ng/ml and about 100 ng/ml, between about 10 ng/ml and about 100 ng/ml, between about 100 ng/ml and about 1000 ng/ml, between about 1 ng/ml and about 10 ng/ml, between about 1 ng/ml and about 5 ng/ml, or between about 1.25 ng/ml and about 5 ng/ml of the sample.

B2. The kit of B or B1, wherein the first and the second amounts of tHTT are determined by an immunoassay comprising the anti-HTT antibody.

B3. The kit of any one of B-B2, wherein the anti-HTT antibody can bind to both wtHTT and mHTT.

B4. The kit of any one of B-B3, wherein the anti-HTT antibody is attached to a label.

B5. The kit of any one of B-B3, further comprising a secondary antibody that binds to the anti-HTT antibody, wherein the secondary antibody is attached to a label.

B6. The kit of any one of B4-B5, wherein the label is a chemiluminescent molecule, a fluorochromes, a colored molecules, a radioisotope, or a combination thereof.

B7. The kit of any one of B-B6, wherein the mHTT-specific antibody composition further comprises particles.

B8. The kit of B7, wherein the particles are agarose particles or magnetic particles.

B9. The kit of any one of B-B8, wherein the mHTT specific antibody is an anti-polyglutamine antibody.

B10. The kit of any one of B7-B9, wherein the particles are linked to the mHTT specific antibody.

B11. The kit of B10, wherein the particles linked to the mHTT specific antibody through antibody binding proteins, streptavidin-biotin interaction, or covalent immobilization.

B12. The kit of B10 or B11, wherein the particles are linked to the mHTT specific antibody through streptavidin-biotin interaction, wherein the particles are attached to streptavidin, and the mHTT specific antibody is in biotinylated form.

B13. The kit of any one of B8-B12, wherein the step (c) further comprises removing the mHTT-specific antibody composition from the sample by centrifugation, wherein the particles are agarose particles.

B14. The kit of any one of B8-B12, wherein the step (c) further comprises removing the mHTT-specific antibody composition from the sample by applying a magnetic field to the sample, wherein the particles are magnetic particles.

B15. The kit of any one of B-B14, wherein the sample is a cerebral spinal fluid sample.

B16. The kit of any one of B-B15, wherein the sample is from a subject having Huntington's disease.

B17. The kit of B16, wherein the subject has received or is receiving a treatment for Huntington's disease.

C. A method of treating a subject, the method comprising:

a) determining the amount of wild type huntingtin protein (wtHTT) in a cerebral spinal fluid sample from the subject, wherein determining comprises:

(i) determining a first amount of total huntingtin protein (tHTT) in the sample;

(ii) contacting the sample with a mutant huntingtin protein (mHTT)-specific antibody composition comprising an mHTT-specific antibody, wherein the concentration of the mHTT-specific antibody is a function of the first amount of the tHTT;

(iii) removing the mHTT-specific antibody composition from the sample; and

(iv) determining the amount of wtHTT in the sample by determining a second amount of tHTT in the sample; and

b) administering to the subject a treatment for Huntington's disease if the amount of wtHTT determined in step (a) is decreased as compared to a reference value.

C1. A method of adjusting a treatment for Huntington's disease of a subject that has received or is receiving the treatment, the method comprising:

a) determining the amount of wild type huntingtin protein (wtHTT) in a cerebral spinal fluid sample from the subject having Huntington's disease, wherein determining comprises:

(i) determining a first amount of total huntingtin protein (tHTT) in the sample;

(ii) contacting the sample with a mutant huntingtin protein (mHTT)-specific antibody composition comprising an mHTT-specific antibody, wherein the concentration of the mHTT-specific antibody is a function of the first amount of the tHTT;

(iii) removing the mHTT-specific antibody composition from the sample; and

(iv) determining the amount of wtHTT in the sample by determining a second amount of tHTT in the sample; and

b) continuing administering the treatment to the subject if the amount of wtHTT determined in step (a) is unchanged or decreased as compared to a reference value.

C2. The method of C or C1, wherein when the first amount of tHTT is at least about 15 fM, the concentration of the mHTT-specific antibody is between about 0.01 ng/ml and about 1000 ng/ml, between about 1 ng/ml and about 100 ng/ml, between about 10 ng/ml and about 100 ng/ml, between about 100 ng/ml and about 1000 ng/ml, between about 1 ng/ml and about 10 ng/ml, between about 1 ng/ml and about 5 ng/ml, or between about 1.25 ng/ml and about 5 ng/ml of the sample.

C3 The method of any one of C-C2, wherein the first and the second amounts of tHTT are determined by an immunoassay comprising an anti-huntingtin protein (anti-HTT) antibody.

C4. The method of C3, wherein the anti-HTT antibody can bind to both wtHTT and mHTT.

C5. The method of 3 or C4, wherein the anti-HTT antibody is attached to a label.

C6. The method of C3 or C4, wherein the immunoassay comprises a secondary antibody that binds to the anti-HTT antibody, wherein the secondary antibody is attached to a label.

C7. The method of any one of C3-C6, wherein the label is a chemiluminescent molecule, a fluorochromes, a colored molecules, a radioisotope, or a combination thereof.

C8. The method of any one of C-7, wherein the mHTT-specific antibody composition further comprises particles.

C9. The method of C8, wherein the particles are agarose particles or magnetic particles.

C10. The method of any one of C-C9, wherein the mHTT specific antibody is an anti-polyglutamine antibody.

C11. The method of any one of C8-C10, wherein the particles are linked to the mHTT specific antibody.

C12. The method of C11, wherein the particles are linked to the mHTT specific antibody through antibody binding proteins, streptavidin-biotin interaction, or covalent immobilization.

C13. The method of C11 or C12, wherein the particles are linked to the mHTT specific antibody through streptavidin-biotin interaction, wherein the particles are attached to streptavidin, and the mHTT specific antibody is in biotinylated form.

C14. The method of any one of C9-C13, wherein the step (a)-(ciii) further comprises removing the mHTT-specific antibody composition from the sample by centrifugation, wherein the particles are agarose particles.

C15. The method of any one of C9-C13, wherein the step (a)-(ciii) further comprises removing the mHTT-specific antibody composition from the sample by applying a magnetic field to the sample, wherein the particles are magnetic particles.

C16. The method of any one of C-C15, wherein the reference value is an amount of wtHTT of subjects not having Huntington's Disease.

### 6. EXAMPLES

The present disclosure will be better understood by reference to the following Examples, which are provided as exemplary of the presently disclosed subject matter, and not by way of limitation.

### Example 1: Novel Immunoassay Schema to Quantify wtHTT in CSF of Huntington's Disease Patients

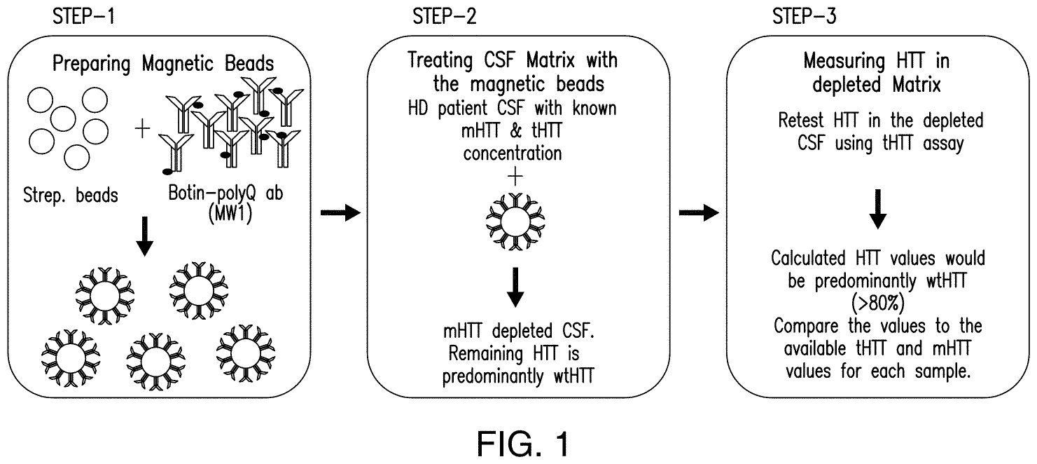

The present disclosure relates to the discovery of a novel assay schema to determine the amount of wtHTT in the CSF of Huntington's disease (HD) patients. In an exemplary embodiment of the presently disclosed methods, patients' CSF samples were initially tested for mHTT and tHTT using immunoassays (FIG. 1). After which, the CSF samples were selectively immunodepleted of mHTT using mHTT specific antibodies. The immunodepleted CSF samples were then re-tested to determine tHTT levels. The re-tested tHTT levels would be the wtHTT levels in the samples because the step of immunodepletion selectively removed mHTT from the samples and left the wtHTT in the samples unaltered. Concentrations of the mHTT specific antibody used in depleting the samples and certain conditions of the assay were optimized to ensure selective and efficient depletion of mHTT from the samples. Additionally, endogenous mHTT and tHTT levels were measured before the immunodepletion step to determine the suitable concentrations of the mHTT specific antibody and for quality control purposes.

### Methods

### Preparation of MW1—Biotin Antibody Conjugates

MW1 antibody was conjugated to biotin using methods known in the art. The MW1 antibody labeled with biotin had a final concentration of 1 μg/μl.

### MW1-Biotin Streptavidin Conjugated to Magnetic Beads (MPs)

Streptavidin (strep) conjugated MPs were procured from a commercial supplier. The required amount of strep-MP for the experiment was put into a separate tube after thoroughly mixing with a pipette. The required amount of biotin conjugated MW1 antibody was then added to the tube and incubated with the strep-MP for 1 hour on a rocker at room temperature. The target concentration of MW1 antibody is 25 μg/mg of MPs. Magnetic beads with MW1 bound using biotin and streptavidin were washed at least 3 times using a PBS tween buffer. After last wash, the beads were re-suspended in PBS and BSA containing buffer.

Mutant HTT (mHTT) Assay

The levels of mHTT in human CSF were measured in triplicate using an SMC™ Erenna® Singluex based immunoassay. Antibodies 2B7 and MW1 were used. The MW1 antibody was polyglutamate chain dependent and 2B7 is independent of polyglutamate (see Fodale V et al, J. Huntington's Disease 2017; 6: 349-361 for details of the mHTT assay).

Hemoglobin concentrations were measured in duplicate using a commercially available ELISA assay to determine the extent of blood contamination by CSF.

Total HTT (tHTT) Assay

The levels of tHTT assay in human CSF were measured in triplicate using an SMC™ Erenna® Singluex based immunoassay. Antibodies 2B7 and DF7 were used. These antibodies were polyglutamate chain independent (see Schill R J et al, Lancet Neurol 2020; 19: 502-12 for details of the tHTT assay).

Hemoglobin concentrations were measured in duplicate using a commercially available ELISA assay to determine the extent of blood contamination by CSF.

Immunoprecipitation of mHTT

At least 400 μL of artificial CSF (aCSF) medium or pooled non-HD human CSF (phCSF) were incubated with MW1-biotin bound Strep-MP with different dilatation ratios (1:50, 1:100, 1:200, 1:300, 1:400 and 1:800), prepared in PBS-BSA buffer. The mixture of diluted Strep-MP and HTT containing media were incubated at least 1 hr on a rocker at room temperature. After the completion of the incubation, MPs were pelleted in the tubes using a tube holder with magnetic strip. The supernatant medium predominantly without mHTT protein was collected into a fresh tube. The supernatant media was reanalyzed for huntingtin concentration using tHTT assay. The resultant HTT levels after pulldown were predominantly wtHTT (e.g., >90% of HTT remaining in the sample was wtHTT).

Assay to measure wtHTT in aCSF

Spike recovery experiments were performed in artificial CSF (aCSF) medium. Various concentrations of full length recombinant HTT proteins with polyglutamine (polyQ) lengths of Q23 or Q48 were used for spike recovery experiments. The spike recovery studies were carried out at various concentrations, including low (15 fM), medium (50 fM), and high (200 fM). In addition, certain spiked samples were prepared with both the polyQ varieties. In these samples the spiked ratio of both the proteins were in 1:1 ratio to keep tHTT concentrations the same

Assay to Measure wtHTT in phCSF

HTT protein levels in non-HD CSF donors were measured using tHTT assay. The CSF samples from these non-HD donors would not have mHTT. CSF samples having similar amounts of HTT were pooled into batches (example 40 fM, 70 fM, etc.). The samples that were in HTT range of 40 fM or higher were selected to move forward with immunoprecipitation. In these batches, known concentrations of recombinant mHTT protein were spiked for the recovery studies. CSF samples that were collected as supernatant after immunoprecipitation would be re-assayed for HTT using tHTT assay. The resultant HTT levels after pulldown were predominantly (>90%) wtHTT.

### Results

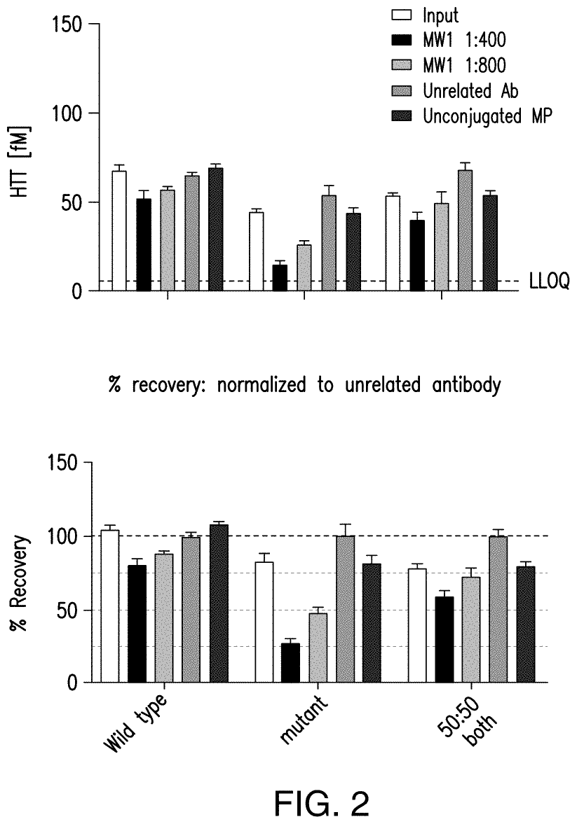

One concentration of HTT protein was spiked in aCSF medium to show dose dependent specificity in pulling down spiked mHTT protein, and to show optimization of magnetic particle used in maximizing pull-down without interfering with the presently disclosed assay (FIG. 2). Three groups of samples were prepared in aCSF medium, where samples of group 1 were spiked with 50 fM recombinant full length wtHTT protein, with 23 Q; samples of group 2 were spiked with 50 fM recombinant full length mHTT protein, with 48 Q; and group 3 with both wtHTT and mHTT in 1:1 ratio at a final concentration of 50 fM. For each group, a set of samples were used as input, and one set each were treated with MW1-Strep-MPs at 1:400 or 1:800, Strep-MP bound to unrelated Ab (control), or blocked Strep-MPs as unconjugated Ab (control). Each sample was tested for HTT in triplicate wells using tHTT assay on SMC Singulex Erenna instrument.

FIG. 2 provides the raw data for HTT in fM (top bar graph) and the same data calculated as % recovery by normalizing to unrelated antibody (bottom bat graph). Control samples, such as inputs and unconjugated Ab are relatively unchanged (≤25% to unrelated Ab) when compare to normalized unrelated Ab samples. Only in samples with mHTT spiked by itself or in combination with wtHTT, the tHTT readings were lowered when treated with 1:400 and 1:800 dilution of the MP complexes. The pull-down percentage was MW1-Strep-MPs concentration dependent, where 1:400 dilution of MW1-Strep-MPs had lower HTT values compared to 1:800 of MW1-Strep-MPs. Lowering of HTT in wtHTT alone spiked samples were relatively unchanged (≤25% to unrelated Ab). Thus, these data suggested that the pulldown was MW1 antibody dependent and was specific to mHTT. HTT measured after pull-down was predominantly wtHTT since mHTT was specifically pulled down in these samples.

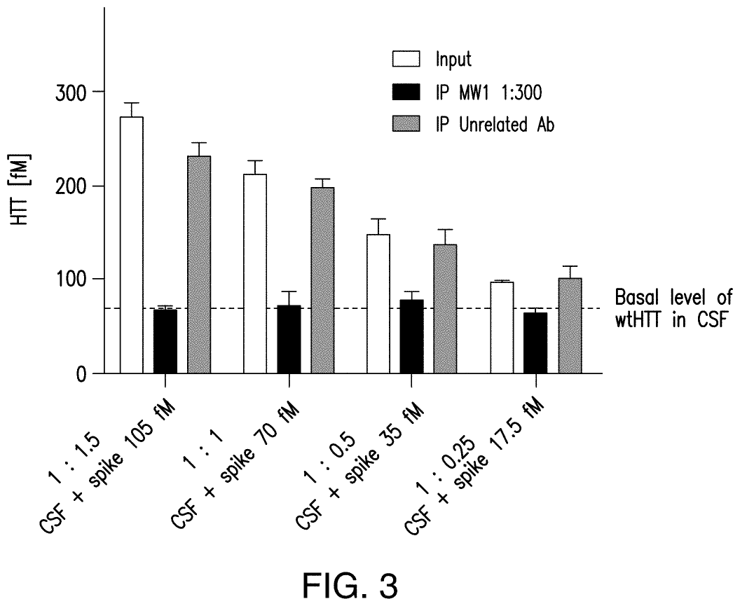

Next, pooled non-HD human CSF (phCSF) with endogenous HTT of approximately 70 fM (expecting all the HTT measured is wild type with no mutant HTT protein because of the donor selection) was used as sample matrix with the baseline wtHTT. Specificity of mHTT pulldown at various concentration ratios of mHTT and optimization of MW1-Strep-MPs dilutions to achieve maximum mHTT pulldown in human CSF without interfering with the endogenous wtHTT were tested. As shown in the FIG. 3, four groups of samples were prepared with phCSF, where group 1 has 1:1.5, group 2 has 1:1, group 3 has 1:0.5, and group 4 has 1:0.025 of wtHTT versus spiked recombinant full length mHTT with 48 polyQ. The resulting concentrations of recombinant mHTT at each group were approximately 105 fM (group 1), 70 fM (group 2), 35 fM (group 3) and 17.5 fM (group 4), respectively. As controls, one set of samples with one from each group was used as input and another set with unrelated Ab for immunoprecipitation (IP) control. The data was plotted as bar graph, with HTT in fM as y axis and various groups on x axis (FIG. 3). In the control samples, HTT concentrations were relatively unchanged (≤30% to unrelated Ab) when compare to unrelated Ab samples. In all groups, when the samples were treated with 1:300 dilution of MW1-Strep-MPs, the HTT values were reduced to endogenous wtHTT levels, showing that the beads pulled down just mHTT and leaving the wtHTT unaltered. The data clearly depicts that the MW1-Strep-MPs specifically pulled down mHTT and was optimized with >90% efficiency of mHTT pull down at HTT concentrations of at least 175 fM in phCSF. The HTT concentration obtained after the mHTT pulldown was determined to be predominantly wtHTT.

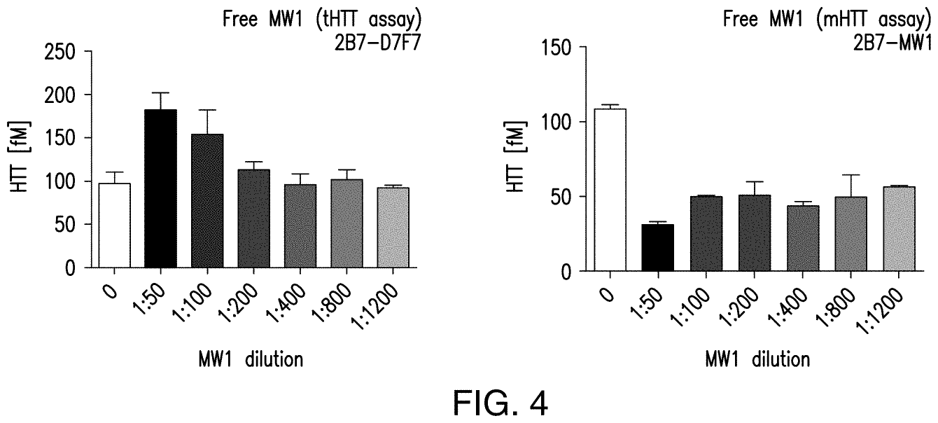

To determine non-specific interactions or interference of free MW1 antibody (used for pulling down mHTT protein) on tHTT assay and mHTT assays on SMC platform developed independently on Singulex Erenna instrument. The experiment was carried out in aCSF, where different dilutions of 1 μg/mL of free MW1 antibody stock was spiked in the aCSF medium with 150 fM nominal recombinant full length mHTT with 48 polyQ spiked and incubated for one hour before tested in two different assays. The MW1 stock dilutions tested were on x axis and HTT raw values in fM on y axis were from tHTT assay (shown in left bar graph of FIG. 4) and from mHTT assay (shown in right bar graph of FIG. 4). In mHTT assay, all dilutions of free MW1 spiked and tested were shown interference, where the HTT values were lower than the untreated (“0” dilution”) sample. The mHTT assay also had MW1 as a detector antibody. Thus, the free MW1 added was captured onto the HTT protein and blocked the detector ab to bind. As the dilution of free MW1 stock solution increased from 1:50 to 1:1200, the HTT signal was partially recovered from 25% to 50% of untreated, as shown in the right bar graph of FIG. 4. However, concentration dependent dose response was not clearly seen, which might be because of tightly bound free MW1 (slow off-rate) and concentration range tested might higher in the plateau region of the competition curve. While in tHTT assay, free MW1 showed no interference when stock tested was 1:200 and further diluted, which was shown in the left bar graph of FIG. 4. A clear interference of HTT quantification was seen up to 1:100 dilution of the MW1 stock. Therefore, based on this data, the MW1-Strep-MPs should be diluted at least 1:200, or higher to avoid possible interference.

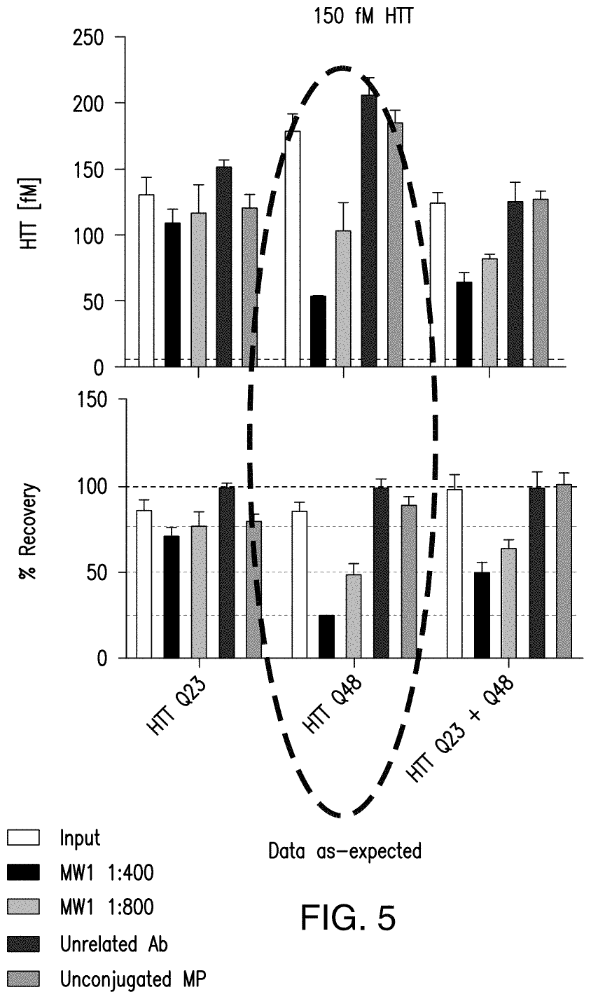

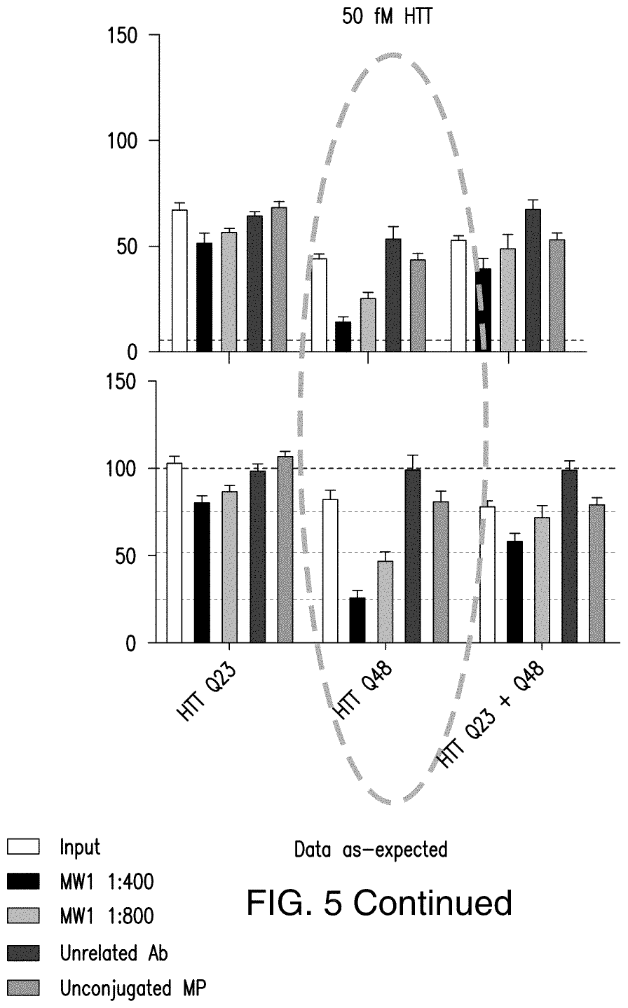

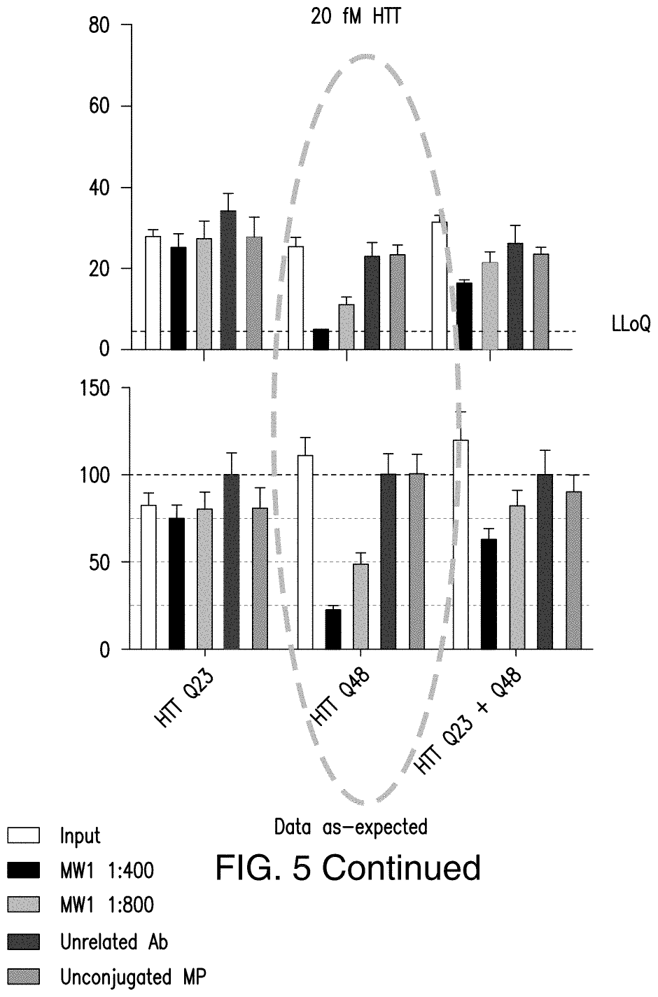

Furthermore, three different concentrations of HTT protein spiked into aCSF medium and tested to show the working range of the assay in a controlled system such as aCSF. The concentrations of recombinant full length wtHTT (23 Q) or mHTT (48 Q) proteins spiked were 20 fM, 50 fM and 150 fM. At each concentration, three groups of samples were prepared. For group 1, samples were spiked with respective fM of recombinant full length wtHT protein, with 23 Q; group 2 samples were spiked with respective fM recombinant full length mHTT protein, with 48 Q; and group 3 with both wtHTT and mHTT in 1:1 ratio at final concentration of respective fM. From each group, a set of samples was used as input, and one set each was treated with MW1-Strep-MPs at 1:400, 1:800, Strep-MP bound to unrelated Ab (control), or blocked Strep-MPs as unconjugated Ab (control). Each sample was tested for HTT in triplicate wells using tHTT assay on SMC Singulex Erenna instrument. The raw data for all three HTT levels (20 fM, 50 fM and 150 fM) are shown in top bar graph of FIG. 5 and the same data calculated as % recovery by normalizing to unrelated antibody is shown in lower bat graph of FIG. 5. Control samples, such as inputs and unconjugated Ab are relatively unchanged (≤25% to unrelated Ab) when compare to normalized unrelated Ab samples in all three HTT levels tested. However, only in samples with mHTT spiked by itself or in combination with wtHTT, the HTT were lowered when treated with 1:400 and 1:800 dilution of the MP complexes. The pull down percentage was MW1-Strep-MPs concentration dependent, where 1:400 dilution with more MW1-Strep-MPs had lower HTT values compared to 1:800 MW1-Strep-MPs with lower MPs. The lowering of HTT in wtHTT alone spiked samples were relatively unchanged (≤25% to unrelated Ab). However, at 20 fM with 1:1 ratio of wtHTT and mHTT treated with 1:400 dilution of MW1-Strep-MPs, the data showed slightly lower than 50% pulled down of mHTT protein. This data confirmed that levels at lower range of the assay (≤20 fM) is possible but the percentage of mHTT pull down is not very efficient. This data shows that in actual patient CSF samples, tHTT values of 20 fM or higher is preferred. MW1 antibody dependent and specific to mHTT. HTT measured after pulldown was predominantly wtHTT since mHTT was specifically pulled down in these samples.

Next, three different concentrations of HTT protein were spiked into aCSF medium. The experiment showed that in the working range of the assay, MW1-Strep-MPs did not interfere with the mHTT protein species present in the sample. The concentrations of recombinant full length wtHTT (23 Q) or mHTT (48 Q) proteins spiked were 20 fM, 50 fM and 150 fM. At each concentration of HTT protein, four groups of samples were prepared. For group 1 samples were spiked with respective fM of recombinant full length wtHT protein, with 23 Q; group 2 samples were spiked with respective fM recombinant full length mHTT protein, with 48 Q; group 3 with both wtHTT and mHTT in 1:1 ratio at final concentration of respective fM; and group 4 with just HTT spiked aCSF was used as a control to test nonspecific interactions to the MPs or MW1 antibodies tested. From each group, a set of samples were used as input, and one set each were treated with MW1-Strep-MPs at 1:400, 1:800, Strep-MP bound to unrelated Ab (control), or blocked Strep-MPs as unconjugated Ab (control). Each sample was tested for mHTT in triplicate wells using mHTT assay on SMC Singulex Erenna instrument.

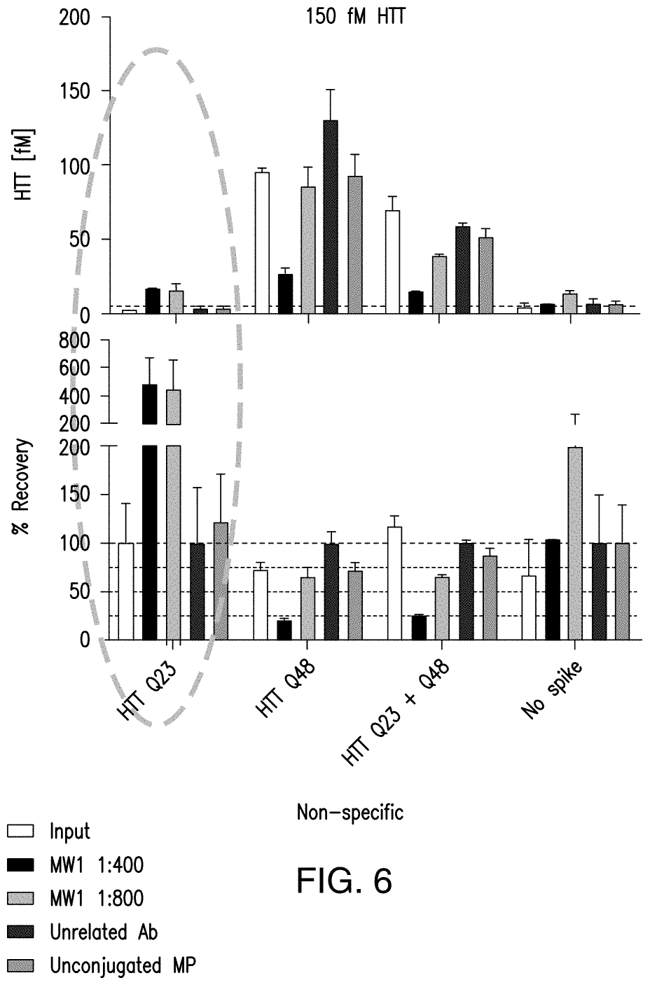

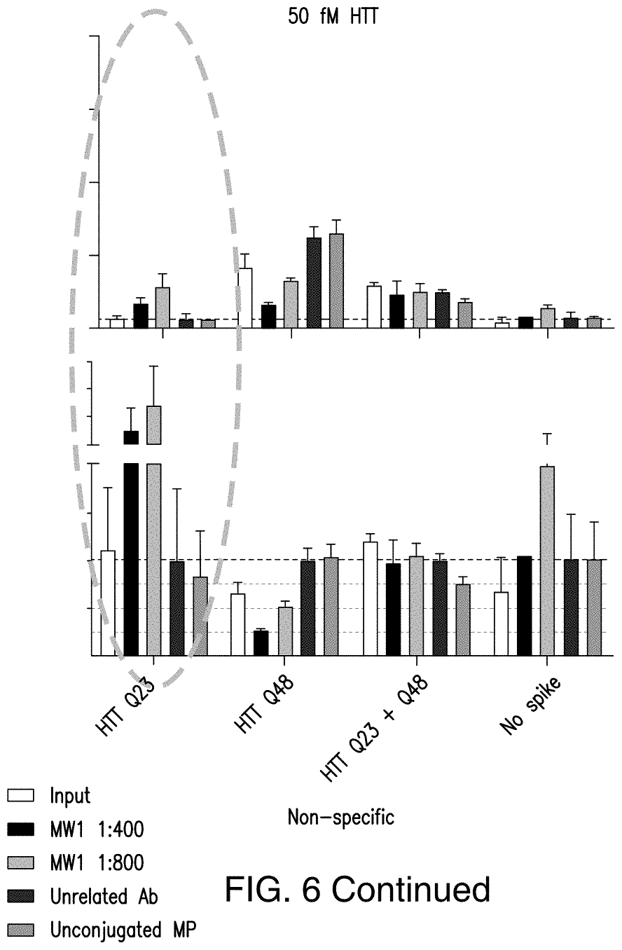

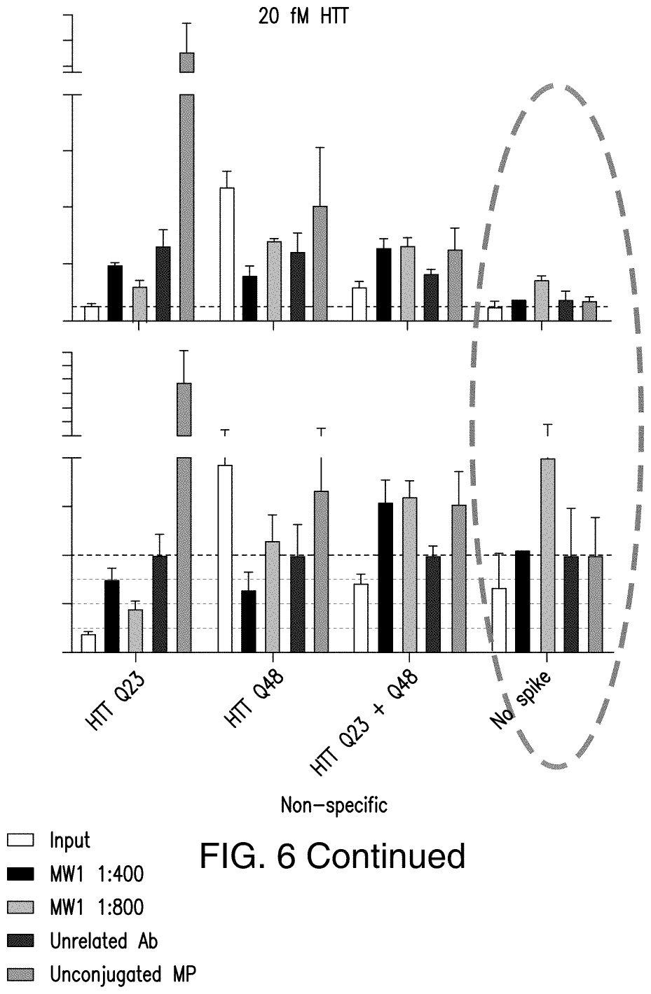

Raw data for all three HTT levels (20 fM, 50 fM and 150 fM) are shown in top bar graph of FIG. 6 and the same data calculated as % recovery by normalizing to unrelated antibody is shown in lower bat graph of FIG. 6. Control samples, such as inputs and unconjugated Ab were relatively unchanged (≤25% to unrelated Ab) when compare to normalized unrelated Ab samples in all three HTT levels tested. However, in wtHTT alone spiked samples, nonspecific signal was shown in both dilutions of MW1-Strep-MPs treated samples. This non-specific signal was consistent with all HTT concentration levels. No signal was expected in any of these samples and importantly in the no-spike controls. This finding showed that the beads or MW1 antibody was interfering with the assay reagents but not the HTT protein. When just mHTT was spiked or 1:1 ratio of wtHTT and mHTT species were spiked into the samples, the results showed that increased MW1-Strep-MPs resulted in more mHTT was pulled down. The non-specific data in the bar graphs was shown in the figure with the dotted line ovals covering the samples.

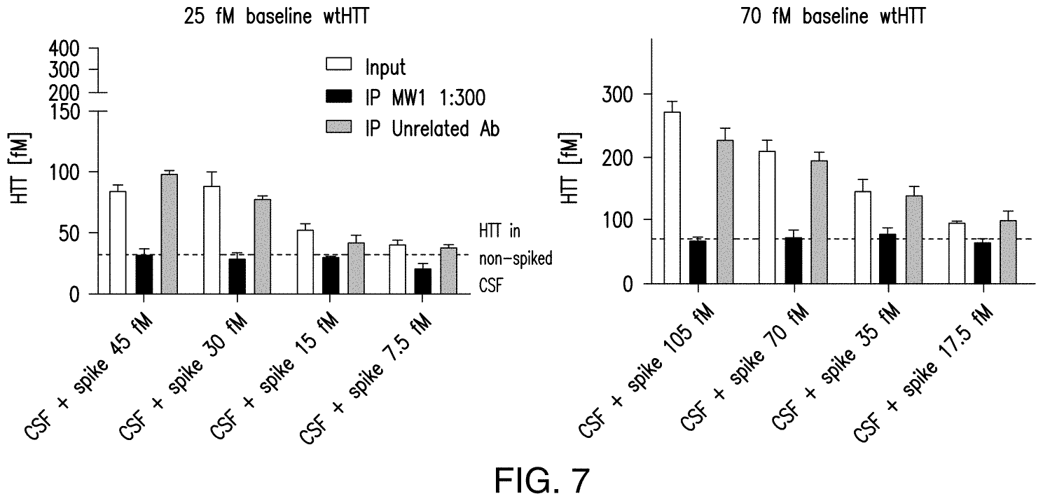

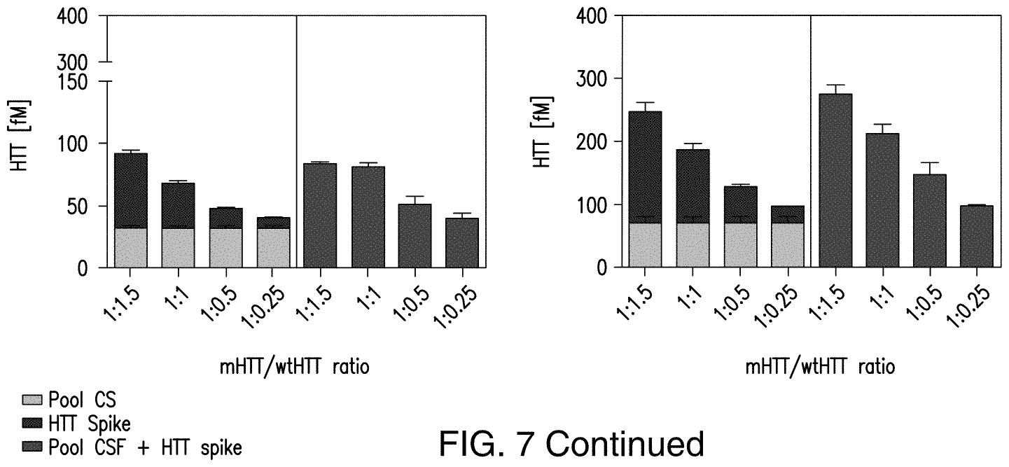

Next, pooled non-HD human CSF (phCSF) with two levels of endogenous HTT of approximately 25 fM and 70 fM (expecting all the HTT measured is wildtype with no mutant HTT protein because samples were from non-HD subjects) was used as sample matrix with these two baseline wtHTT values at each level tested. In this experiment working range of HTT values with mHTT pulldown at various concentration ratios were tested in pooled human CSF without interfering with the endogenous wtHTT. FIG. 7 showed both the levels of endogenous wtHTT tested in four groups of samples that were prepared in phCSF. Group 1 has 1:1.5, group 2 has 1:1, group 3 has 1:0.5, and group 4 has 1:0.025 of wtHTT versus spiked recombinant full length mHTT with 48 polyQ. The resulting concentrations of recombinant mHTT at each group were approximately 105 fM (group 1), 70 fM (group 2), 35 fM (group 3) and 17.5 fM (group 4), respectively. The concentrations were listed on the x axis and the HTT values in fM were plotted on y axis of the bar graphs. The top bar graphs of FIG. 7 showed unnormalized data and the same data was plotted as a ratio of mHTT to wtHTT (endogenous wtHTT value) as shown in the lower bar graphs of FIG. 7.

As controls, one set of samples with one from each group was used as input and another set with unrelated Ab for immunoprecipitation (IP) control. In the control samples for both levels, HTT concentrations were relatively unchanged (≤30% to unrelated Ab) when compared to unrelated Ab samples (FIG. 7). In all groups of both the wtHTT levels, when the samples were treated with 1:300 dilution of MW1-Strep-MPs, the HTT values dropped down to endogenous wtHTT levels, showing that the beads pulled down just mHTT and leaving the wtHTT unaltered (FIG. 7). The assay was repeated three times to test reproducibility. The data shared here (FIG. 7) is one of the three independent runs. These data clearly showed that the MW1-Strep-MPs specifically pulled down mHTT and the conditions were optimized with >90% efficiency of mHTT pull-down with HTT concentrations at both levels (25 fM and 70 fM) in phCSF. The HTT concentration obtained after pull-down was determined to be predominantly wtHTT.

### Example 2: Measuring wtHTT in the CSFs of HD Patients

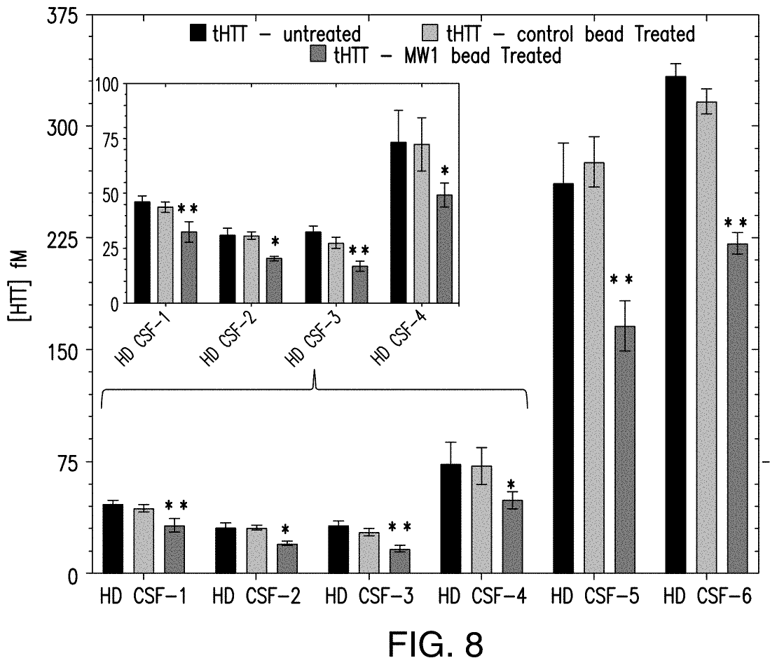

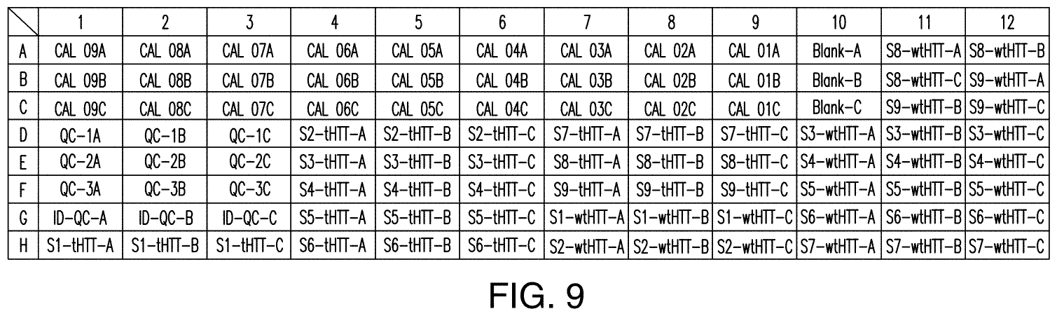

The assay optimized in buffer and non-HD CSF was used with same conditions to measure wtHTT in HD disease patient CSF disclosed in Example 1. For which six individual patient CSFs were tested for wtHTT and tHTT on the optimized assay. Each individual CSF sample was listed as “HD CSF”-1 through 6. As expected in all six individuals wtHTT were shown to be lower than tHTT (FIG. 8). While testing the samples four levels of QCs were also tested. The QC concentrations were selected ranging between lower level of quantification (LLOQ=20 fM) and upper level of quantification (ULOQ=1500 fM) to check assay performance during the HD sample testing. In addition to the four QCs, a fifth QC with 1:1 ratio of recombinant full length wtHTT and full length mHTT were also tested to check performance of immune depletion step while testing the HD samples. Enlarged image of the first four HD CSF samples were shown in the inset of graph provided (FIG. 8). In all six HD CSF tested the percentage of wtHTT is >50% of the tHTT.tHTT. An example of 96 well plate map is shown in a figure (FIG. 9) with calibrators (CAL 0 #), QCs (QC 0 #), immunodepletion QC (ID-QC), samples for tHTT (S #-tHTT), and samples for wtHTT (S #-wtHTT), where “#” represents numbers in replicates indicated as alphabets A, B, or C. The arrangement on a 96 well can be in any fashion.

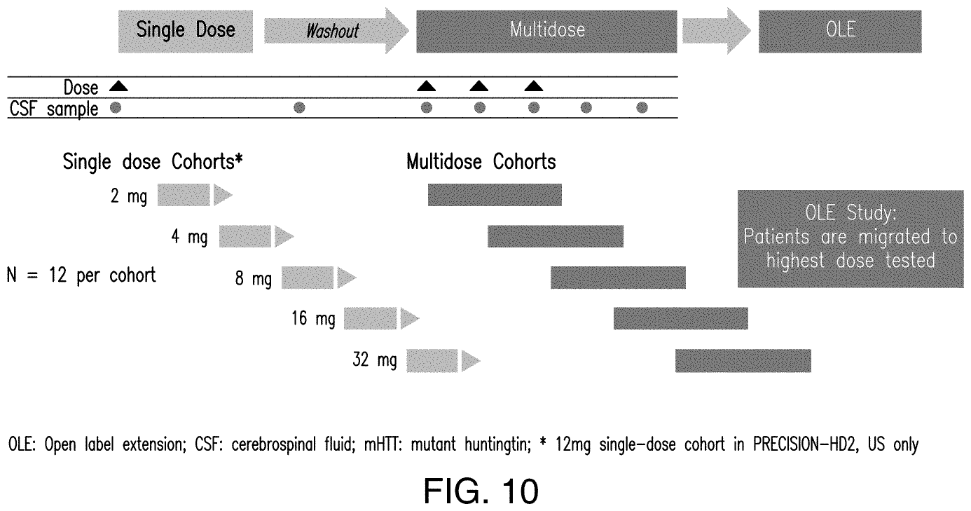

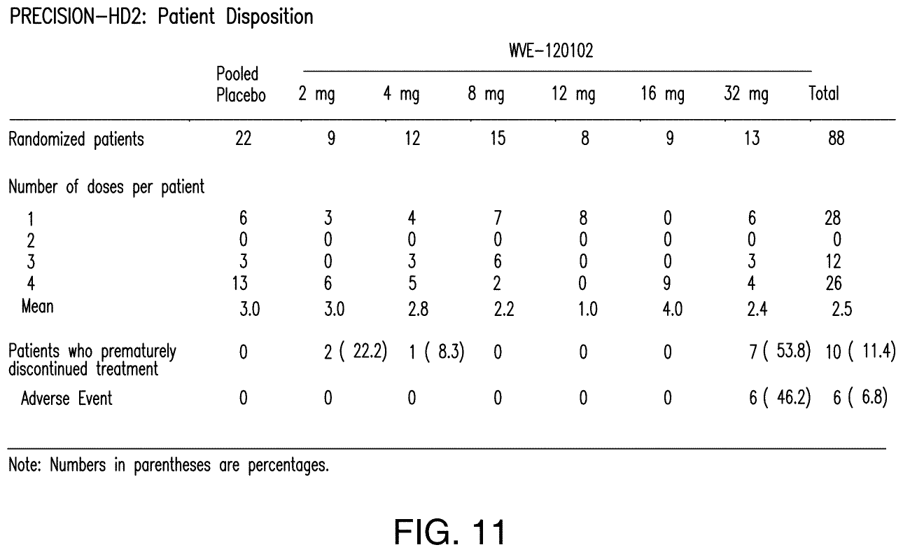

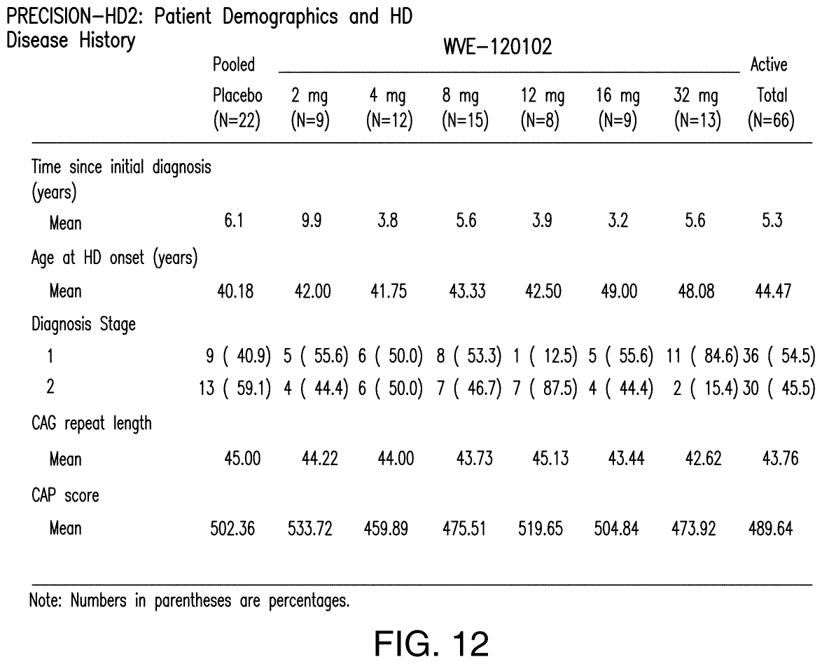

Two PRECISION-HD studies were conducted, PRECISION-HD1 and PRECISION-HD2, to examine the effects of an HD treatment. The study design of the PRECISION-HD studies is shown in FIG. 10. The patient dispositions of PRECISION-HD2 study are shown in FIG. 11, and the patient demographics and HD disease history of PRECISION-HD2 study are shown in FIG. 12. wtHTT levels were measured in CSF samples collected from HD patients using the presently disclosed methods, for examples, the methods disclosed in Example 1.

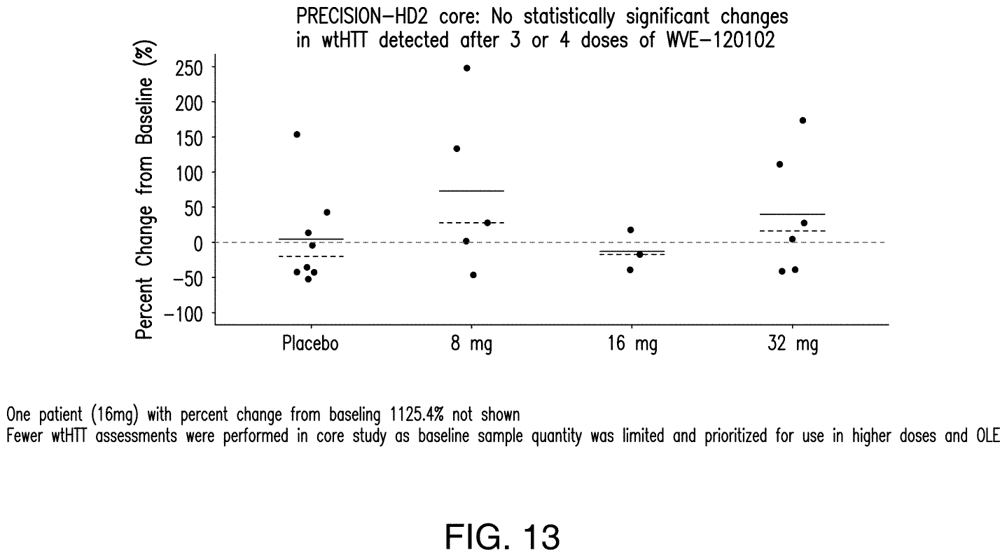

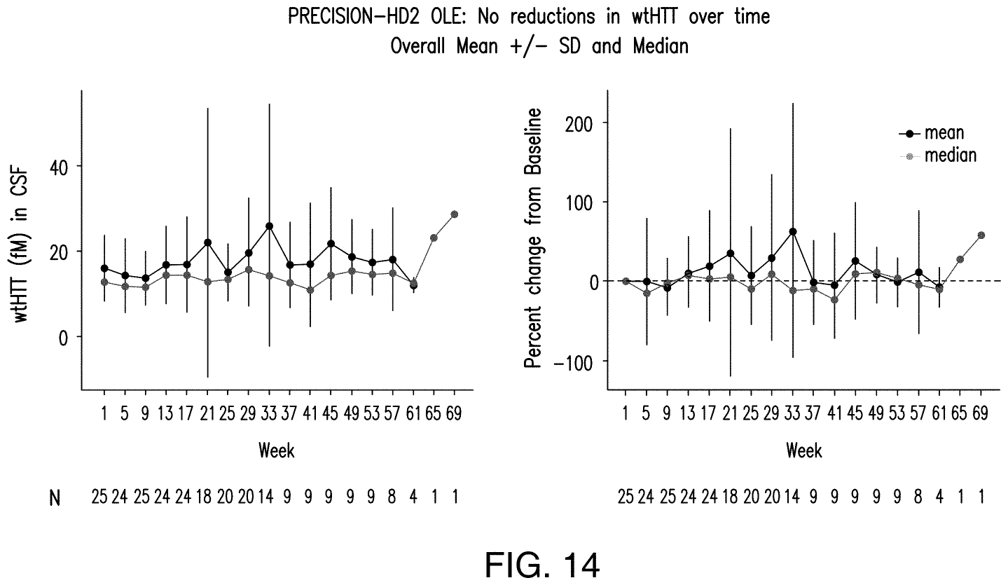

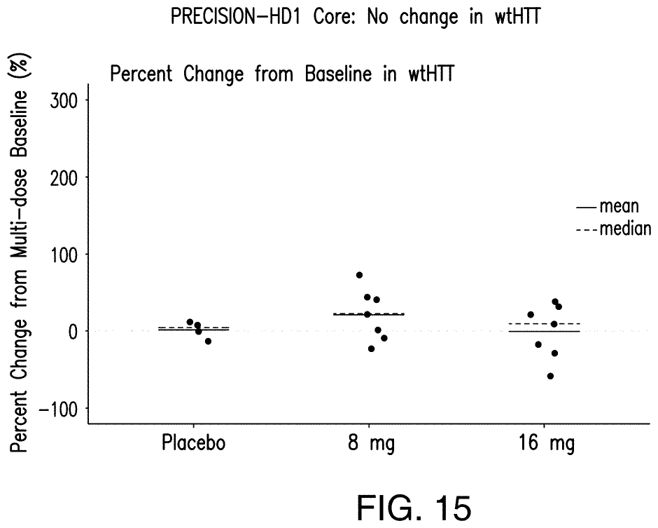

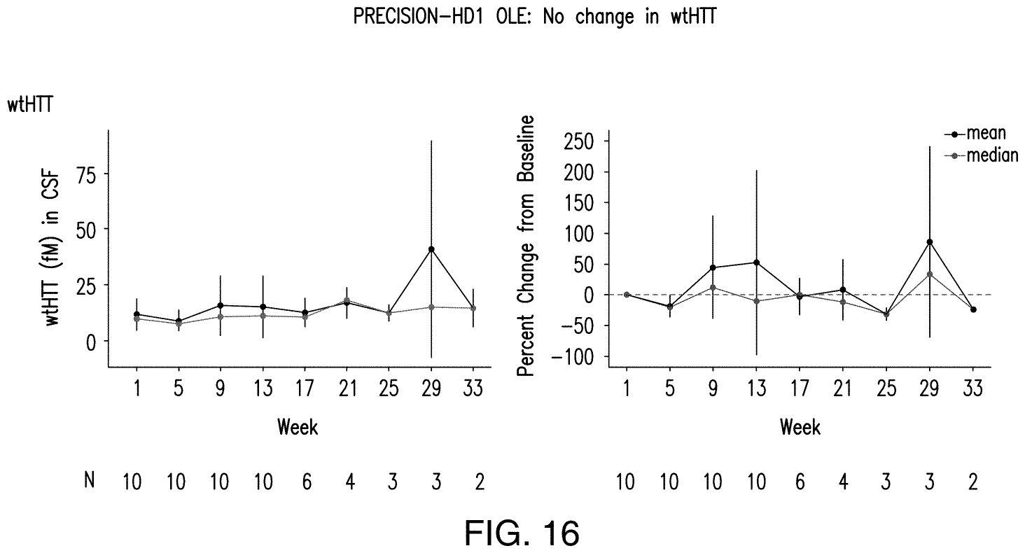

No statistically significant changes in wtHTT were detected after 3 or 4 doses of the HD treatment in PRECISION-HD2 core study (FIG. 13). In the PRECISION-HD2 OLE study, no reduction in wtHTT was detected over time (FIG. 14). Similar results were obtained from the PRECISION-HD1 core study and OLE study (FIGS. 15 and 16).

Although the present disclosure and certain of its advantages have been described in detail, it should be understood that various changes, substitutions and alterations can be made herein without departing from the spirit and scope of the disclosure. Moreover, the scope of the present application is not intended to be limited to the particular embodiments of the process, machine, manufacture, and composition of matter, and methods described in the specification. As one of ordinary skill in the art will readily appreciate from the disclosure of the presently disclosed subject matter, processes, machines, manufacture, compositions of matter, or methods, presently existing or later to be developed that perform substantially the same function or achieve substantially the same result as the corresponding embodiments described herein may be utilized according to the presently disclosed subject matter. Accordingly, the appended claims are intended to include within their scope such processes, machines, manufacture, compositions of matter, or methods.

Various patents, patent applications, publications, product descriptions, protocols, and sequence accession numbers are cited throughout this application, the disclosure of which are incorporated herein by reference in their entireties for all purposes.

What is claimed is:

1. A method of determining the amount of wild type huntingtin protein (wtHTT) in a sample, the method comprising:
(a) determining a first amount of total huntingtin protein (tHTT) in the sample;
(b) contacting the sample with a mutant huntingtin protein (mHTT)-specific antibody composition comprising an mHTT-specific antibody, wherein the concentration of the mHTT-specific antibody is a function of the first amount of the tHTT;
(c) removing the mHTT-specific antibody composition from the sample; and
(d) determining the amount of wtHTT in the sample by determining a second amount of tHTT in the sample.

2. The method of claim 1, wherein when the first amount of tHTT is at least about 15 fM, the concentration of the mHTT-specific antibody is between about 0.01 ng/ml and about 1000 ng/ml, between about 1 ng/ml and about 100 ng/ml, between about 10 ng/ml and about 100 ng/ml, between about 100 ng/ml and about 1000 ng/ml, between about 1 ng/ml and about 10 ng/ml, between about 1 ng/ml and about 5 ng/ml, or between about 1.25 ng/ml and about 5 ng/ml of the sample.

3. The method of claim 1, wherein the first and the second amounts of tHTT are determined by an immunoassay comprising an anti-huntingtin protein (anti-HTT) antibody that can bind to both wtHTT and mHTT.

4. The method of claim 3, wherein the anti-HTT antibody is attached to a label.

5. The method of claim 3, wherein the immunoassay comprises a secondary antibody that binds to the anti-HTT antibody, wherein the secondary antibody is attached to a label.

6. The method of claim 1, wherein the mHTT-specific antibody composition further comprises particles.

7. The method of claim 6, wherein the particles are linked to the mHTT specific antibody through antibody binding proteins, streptavidin-biotin interaction, or covalent immobilization.

8. The method of claim 6, wherein the step (c) further comprises removing the mHTT-specific antibody composition from the sample by centrifugation, wherein the particles are agarose particles, or removing the mHTT-specific antibody composition from the sample by applying a magnetic field to the sample, wherein the particles are magnetic particles.

9. A kit for of determining the amount of wild type huntingtin protein (wtHTT) in a sample, the kit comprising:
a mutant huntingtin protein (mHTT)-specific antibody composition comprising an mHTT-specific antibody;
an anti-huntingtin protein (anti-HTT) antibody; and
instructions for of determining the amount of wild type huntingtin protein (wtHTT) in the sample, wherein the instructions comprise directions to (a) determining a first amount of total huntingtin protein (tHTT) in the sample; (b) contacting the sample with a mutant huntingtin protein (mHTT)-specific antibody composition, wherein the concentration of the mHTT-specific antibody is a function of the first amount of the tHTT; (c) removing the mHTT-specific antibody composition from the sample; and (d) determining the amount of wtHTT protein in the sample by determining a second amount of tHTT in the sample.

10. The kit of claim 9, wherein when the first amount of tHTT is at least about 15 fM, the concentration of the mHTT-specific antibody is between about 0.01 ng/ml and about 1000 ng/ml, between about 1 ng/ml and about 100 ng/ml, between about 10 ng/ml and about 100 ng/ml, between about 100 ng/ml and about 1000 ng/ml, between about 1 ng/ml and about 10 ng/ml, between about 1 ng/ml and about 5 ng/ml, or between about 1.25 ng/ml and about 5 ng/ml of the sample.

11. The kit of claim 9, wherein the first and the second amounts of tHTT are determined by an immunoassay comprising the anti-HTT antibody.

12. The kit of claim 9, further comprising a secondary antibody that binds to the anti-HTT antibody, wherein the secondary antibody is attached to a label.

13. A method of treating a subject, the method comprising:
a) determining the amount of wild type huntingtin protein (wtHTT) in a cerebral spinal fluid sample from the subject, wherein determining comprises:
(i) determining a first amount of total huntingtin protein (tHTT) in the sample;
(ii) contacting the sample with a mutant huntingtin protein (mHTT)-specific antibody composition comprising an mHTT-specific antibody, wherein the concentration of the mHTT-specific antibody is a function of the first amount of the tHTT;
(iii) removing the mHTT-specific antibody composition from the sample; and
(iv) determining the amount of wtHTT in the sample by determining a second amount of tHTT in the sample; and
b) treating the subject with a treatment for Huntington's disease if the amount of wtHTT determined in step (a) is decreased as compared to a reference value.

14. A method of adjusting a treatment for Huntington's disease of a subject that has received or is receiving a treatment, the method comprising:
a) determining the amount of wild type huntingtin protein (wtHTT) in a cerebral spinal fluid sample from the subject having Huntington's disease, wherein determining comprises:
(i) determining a first amount of total huntingtin protein (tHTT) in the sample;
(ii) contacting the sample with a mutant huntingtin protein (mHTT)-specific antibody composition comprising an mHTT-specific antibody, wherein the concentration of the mHTT-specific antibody is a function of the first amount of the tHTT;
(iii) removing the mHTT-specific antibody composition from the sample; and
(iv) determining the amount of wtHTT in the sample by determining a second amount of tHTT in the sample; and
b) continuing administering the treatment to the subject if the amount of wtHTT determined in step (a) is unchanged or decreased as compared to a reference value.

15. The method of claim 13, wherein when the first amount of tHTT is at least about 15 fM, the concentration of the mHTT-specific antibody is between about 0.01 ng/ml and about 1000 ng/ml, between about 1 ng/ml and about 100 ng/ml, between about 10 ng/ml and about 100 ng/ml, between about 100 ng/ml and about 1000 ng/ml, between about 1 ng/ml and about 10 ng/ml, between about 1 ng/ml and about 5 ng/ml, or between about 1.25 ng/ml and about 5 ng/ml of the sample.

16. The method of claim 13, wherein the first and the second amounts of tHTT are determined by an immunoassay comprising an anti-huntingtin protein (anti-HTT) antibody that can bind to both wtHTT and mHTT.

17. The method of claim 16, wherein the anti-HTT antibody is attached to a label.

18. The method of claim 16, wherein the immunoassay comprises a secondary antibody that binds to the anti-HTT antibody, wherein the secondary antibody is attached to a label.

19. The method of claim 13, wherein the mHTT-specific antibody composition further comprises particles.

20. The method of claim 19, wherein the step (a)-(iii) further comprises removing the mHTT-specific antibody composition from the sample by centrifugation, wherein the particles are agarose particles, or removing the mHTT-specific antibody composition from the sample by applying a magnetic field to the sample, wherein the particles are magnetic particles.
